(19) United States
(12) Patent Application Publication
(10) Pub. No.: US 2023/0212317 A1
(43) Pub. Date: Jul. 6, 2023

(54) HETERODIMERIC PROTEINS

(71) Applicant: PETMEDIX LTD, Babraham, Cambridge (GB)

(72) Inventors: Meng Amy LI, Cambridge (GB); Marco BARDELLI, Cambridge (GB); Roberto BANDIERA, Cambridge (GB)

(21) Appl. No.: 17/996,728

(22) PCT Filed: Apr. 21, 2021
(86) PCT No.: PCT/GB2021/050958

§ 371 Date: Oct. 20, 2022

(30) Foreign Application Priority Data
Apr. 22, 2020 (GB) 2005879.8

(51) Int. Cl. C07K 16/46; C07K 16/28

(52) U.S. Cl. CPC C07K 16/46; C07K 16/2809; C07K 2317/31; C07K 2317/526; C07K 2317/622; C07K 2317/94

(57) ABSTRACT

The invention relates to antibody therapies in veterinary medicine, in particular full-length antibodies and antibody fragments, by providing methods to improve the production of heterodimeric antibodies for veterinary medicine as well as providing related products and uses.

### INTRODUCTION

Antibody-based therapeutics have emerged as important components of therapies for an increasing number of human malignancies in such fields as oncology, inflammatory and infectious diseases. Indeed, antibodies are one of the best-selling classes of drugs today; five of the top ten best selling drugs are antibodies. Increasingly, antibody therapy is also used in veterinary medicine for the treatment of domestic animals, such as dogs.

There is a huge need for these therapies in veterinary medicine, as in the USA alone there are 6M cases of cancer diagnosed each year in dogs, and a similar number in cats (Cekanova and Rathore Animal models and therapeutic molecular targets of cancer: utility and limitations. Drug Des Devel Ther, 8:1911-2, 2014) “Animal models and therapeutic molecular targets of cancer: utility and limitations” Drug design, development and therapy 8:1911-1922). Moreover, one in four American dogs is diagnosed with some form of arthritis (Bland “Canine osteoarthritis and treatments: a review” Veterinary Science Development 5(2)), 2015)). Thus, there is potential for application of antibody therapeutics to many chronic veterinary diseases. Monoclonal antibodies could also be beneficial for the detection, prevention and control of parasitic, bacterial and viral diseases.

The first monoclonal antibody for therapeutic use in humans received marketing approval 25 years ago, 80 have since been approved and more than 50 are in late-stage clinical development. In contrast, the use of antibodies in veterinary medicine is in its early stages with just a few antibodies under development. The limited progress reflects the fact that developing species-specific therapeutic antibodies is technically challenging and only a relatively recent endeavour. There is therefore a need to develop improved antibodies for veterinary medicine, as well as methods for making antibodies for veterinary medicine.

Antibody structure has been exploited to engineer a variety of different antibody formats to target disease in humans. An example of such an engineered antibody format is bispecific antibodies. Bispecific antibodies bind to two different targets and are therefore capable of simultaneously binding to two different epitopes. One area of interest is T cell directed bispecific antibodies for efficient tumour killing. Bispecific antibodies can have “two-target” functionality and bind to two different surface receptor or ligands thus influencing multiple disease pathways. Bispecific antibodies can also place two targets in close proximity, either to support protein complex formation on one cell or to trigger contact between cells. Bispecific antibodies formats vary in many ways including their molecular weight, number of antigen-binding sites, spatial relationship between different binding sites, valency for each antigen, ability to support secondary immune functions and pharmacokinetic half-life. These diverse formats provide great opportunity to tailor the design of bispecific antibodies to match the proposed mechanisms of action and the intended clinical application (Kontermann and Brinkmann Bispecific Antibodies Drug Discovery Today Volume 20, Number 7, 2015).

Production of bispecific antibodies of the IgG type by co-expression of the two light and two heavy chains in a single host cell can be highly challenging because of the low yield of desired bispecific IgGs and the difficulty in removing closely related mispaired IgG contaminants. This reflects that heavy chains form homodimers as well as the desired heterodimers—the so-called heavy chain-pairing problem. Additionally, light chains can mispair with non-cognate heavy chains—the so-called light chain pairing problem. Consequently, coexpression of two antibodies can give rise to up to nine unwanted IgG species in addition to the desired bispecific antibody.

Various approaches are described in the art in order to promote heterodimerisation, i.e. the formation of a certain bispecific antibody of interest for human therapy, thereby reducing the content of undesired homodimers in the resulting mixture. These approaches have been studied in relation to human or humanised antibodies designed to target disease in humans.

The homodimerisation of the two heavy chains in an IgG is mediated by the non-covalent interaction between the CH3 domains alone. Thus, CH3-CH3 interaction is the primary driver for Fc dimerisation.

It is furthermore well-known that when two CH3 domains interact with each other they meet in a protein-protein interface which comprises “contact” residues (also called contact amino acids, interface residues or interface amino acids). Contact amino acids of a first CH3 domain interact with one or more contact amino acids of a second CH3 domain. Contact amino acids are typically within 5.5 Å (preferably within 4.5 Å) of each other in the three-dimensional structure of an antibody. The interaction between contact residues from one CH3 domain and contact residues from a different CH3 domain may for instance be via Van der Waals forces, hydrogen bonds, water-mediated hydrogen bonds, salt bridges or other electrostatic forces, attractive interactions between aromatic side chains, disulfide bonds, or other forces known to one skilled in the art. The primary drive is thus hydrophobic interaction in the core and electrostatic interactions.

Approaches to interfere with the dimerisation of antibody heavy chains have been employed in the art to bias production of heterodimeric antibodies. Specific engineering in the CH3 domains was applied in order to favour heterodimerisation over homodimerisation. Examples of such engineering of the CH3-CH3 interface include the introduction of complementary protuberance and cavity mutations, also known as ‘knob-into-hole’ approaches as described for instance in WO9627011 and J. B. Ridgway et al ‘Knobs-into-holes’ engineering of antibody CH3 domains for heavy chain heterodimerisation Protein Eng., 9 (1996), pp. 617-621.

Generally, the method involves introducing a protuberance at the interface of a first polypeptide and a corresponding cavity in the interface of a second polypeptide, such that the protuberance can be positioned in the cavity so as to promote heteromultimer formation and hinder homomultimer formation. “Protuberances” or “knobs” are constructed by replacing small amino acid side chains from the interface of the first polypeptide with larger side chains (e.g. tyrosine or tryptophan). Compensatory “cavities” or “holes” of identical or similar size to the protuberances are created in the interface of the second polypeptide by replacing large amino acid side chains with smaller ones (e.g. alanine or threonine). The protuberance and cavity can be made by synthetic means such as altering the nucleic acid encoding the polypeptides or by peptide synthesis. Starting from a “knob” mutation (T366W) (Ridgway et al., supra) that disfavors CH3 homodimerisation, compensating “hole” mutations (T366S, L368A, and Y407V) (Atwell et al Stable heterodimers from remodeling the domain interface of a homodimer using a phage display library J. Mol. Biol., 270, pp. 26-35, 1997) were identified by phage display providing efficient pairing with the “knob” while disfavoring homodimerisation.

Several other successful strategies for heavy chain heterodimerisation, including electrostatic steering mutations (WO2006/106905 and Gunasekaran et al Enhancing antibody Fc heterodimer formation through electrostatic steering effects: applications to bispecific molecules and monovalent IgG J. Biol. Chem., 285, pp. 19637-19646, 2010). This approach is based on electrostatic engineering of contact residues within the CH3-CH3 interface that are naturally charged. Mutations are introduced in the CH3 domains of heavy chains wherein naturally occurring charged amino acid contact residues are replaced by amino acid residues of opposite charge (i.e. a charge reversal strategy). This creates an altered charge polarity across the Fc dimer interface such that co-expression of electrostatically matched Fc chains support favorable attractive interactions thereby promoting desired Fc heterodimer formation, whereas unfavorable repulsive charge interactions suppress unwanted Fc homodimer formation.

It has been described that within the human CH3-CH3 interface four unique charges residue pairs are involved in the domain-domain interaction. These are D356/K439′, E357/K370′, K392/D399′ and D399/K409′ (numbering according to Kabat, 1991) where residues in the first chain are separated from residues in the second chain by ‘/’ and where the prime (′) indicates the residue numbering in the second chain). As the CH3-CH3 interface displays a 2-fold symmetry, each unique charge pair is represented twice in intact IgG (i.e., also K439/D356′, K370/E357′, D399/K392′ and K409/D399′ charge interactions are present in the interface). Taking advantage of this two-fold symmetry, it was demonstrated that a single charge reversion, e.g. K409D in the first chain, or D399′K in the second chain resulted in diminished homodimer formation due to repulsion of identical charges. Combining different charge reversions further enhanced this repulsive effect. It was demonstrated that expression of different CH3 domains comprising different, complementary charge reversions, could drive heterodimerisation, resulting in an increased proportion of the bispecific species in the mixture (for review see Kontermann and Brinkmann, supra; Brinkmann and Kontermann: The making of bispecific antibodies, MABS, Vol. 9, No. 2, 2017, pages 182-212, Ha et al, Frontiers in Immunology Immunoglobulin Fc Heterodimer Platform Technology: From Design to Applications in Therapeutic Antibodies and Proteins, Vol 7, article 394, 2016).

There is a need to develop improved antibodies for veterinary medicine, as well as methods for making antibodies for veterinary medicine. The invention is aimed at addressing this need, in particular by providing methods to improve the production of heterodimeric antibodies for veterinary medicine as well as providing related products and uses.

### SUMMARY OF THE INVENTION

The invention is based on the finding that mutating certain residues in the canine CH3 domains promotes the formation of heterodimers for the production of bispecific antibodies for veterinary use.

The inventors have also surprisingly identified a conserved canine charge pair in the canine IgG CH3 domain which is highly conserved across all canine IgG isotypes and uncharged in the corresponding positions in all human IgG. The inventors have shown that modifying this charge pair can promote heterodimerisation.

The inventors have modified a canine IgG CH3 domain interface of the Fc region with mutations of charge pairs so that the engineered CH3 containing proteins preferentially form heterodimers. This minimises the formation of homodimer contaminants for the production of bispecific antibodies. Without wishing to be bound by theory, the inventors believe that the mutations created altered charge polarity across the Fc dimer interface such that coexpression of electrostatically matched Fc chains support favourable attractive interactions, thereby promoting desired Fc heterodimer formation, whereas unfavourable repulsive charge interactions suppress unwanted Fc homodimer formation.

Typically, in the various embodiments of the invention, a first canine IgG CH3 domain and a second canine IgG CH3 domain are both engineered in a complementary manner so that each CH3 domain (or a polypeptide comprising it) substantially does not homodimerise with itself or homodimerises at a lower rate, but is forced to heterodimerise with the complementary engineered other CH3 domain. In other words, the first and second CH3 domain heterodimerise and few homodimers between the two first or the two second CH3 domains are formed.

The methods of the invention therefore involve substituting at least one amino acid in a canine IgG CH3 domain of a first polypeptide, and/or substituting at least one amino acid of a canine IgG CH3 domain of the second polypeptide, wherein said amino acid of the CH3 domain of a first polypeptide is facing the at least one amino acid of the CH3 domain of the second heavy chain within the tertiary structure of the heterodimeric protein, for example antibody, wherein the respective amino acids within the CH3 domains of the first and second heavy chain, respectively, are substituted such that amino acids of opposite side chain charges are introduced into the opposing polypeptides. The invention also relates to polypeptides obtained or obtainable by such methods.

The invention also relates to a modified CH3 domain and to a heterodimeric protein or polypeptide comprising a modified CH3 domain, e.g. an isolated CH3 domain, an isolated polypeptide, or heterodimeric protein comprising a modified CH3 domain. The heterodimeric protein comprises a CH3 domain in a first polypeptide and CH3 domain in a second polypeptide, characterized in that the first CH3 domain of the first polypeptide and the second CH3 domain of the second polypeptide each meet at an interface which comprises an original interface between the CH3 domains, wherein said interface is altered to promote the formation of the heterodimeric protein.

There are several isoforms in dog, IgG-A, IgG-B, IgG-C and IgG-D. The aspects of the invention relate to all isoforms. Various embodiments are set out specifically for IgG-D and IgG-B. However, the embodiments also extend to those covering other isoforms where a residue at a corresponding position (corresponding to a position as described for IgG-D and/or IgG-B) is substituted. FIG. 6 shows an alignment of the different isoforms in dog isoforms. The skilled person would be able to identify the corresponding position and residue in a different canine IgG isoforms by aligning the sequences.

Therefore, in a first aspect, the invention relates to a heterodimeric protein comprising

a) a first polypeptide comprising a first canine IgG CH3 domain and

b) a second polypeptide comprising a second canine IgG CH3 domain

wherein said first and second canine IgG CH3 domain comprise one or more charge pair substitution.

The one or more charge pair substitution promotes heterodimerisation.

In a second aspect, the invention relates to a heterodimeric protein comprising

a) a first polypeptide comprising a first canine IgG CH3 domain and

b) a second polypeptide comprising a second canine IgG CH3 domain

wherein only one of the canine IgG CH3 domains comprises one or more charge pair substitution but the other does not. For example, the first canine IgG CH3 domain has a substitution, but the second does not. In another example, the second canine IgG CH3 domain has a substitution, but the first does not.

In a third aspect, the invention relates to a heterodimeric protein comprising a first polypeptide comprising a first canine IgG CH3 domain and a second polypeptide comprising a second canine IgG CH3 domain wherein

a) said first canine IgG CH3 domain comprises an amino acid substitution at one or more of position 424, 423, 377, 378, 382, with an amino acid of the opposite charge and said second canine IgG CH3 domain comprises an amino acid substitution at one or more of position 414, 416, 433, 392, 377, 393 with an amino acid of the opposite charge, an amino acid substitution at 463 with a charged amino acid or with an amino acid of the opposite charge and/or an amino acid substitution at 425 with N;

b) wherein the first canine IgG CH3 domain comprises an amino acid substitution at 383, 393 and/or 382 and the second canine IgG CH3 domain does not comprise a corresponding mutation or

c) wherein the second canine IgG CH3 domain comprises an amino acid substitution at 463 or 425 and the first canine IgG CH3 domain does not comprise a corresponding mutation.

The IgG may be IgG-A, B, C or D. The above numbering refers to IgG-D, but a corresponding position in another isoform may be substituted.

The one or more amino acid substitution promotes heterodimerisation.

For example, a) said first canine IgG CH3 domain comprises an amino acid substitution at one or more of position E424, D423, D/K377, E378 with an amino acid of the opposite charge and said second canine IgG CH3 domain comprises an amino acid substitution at one or more of position K414, H/R416, K433, K392 with an amino acid of the opposite charge and/or an amino acid substitution at L/K463 with a charged amino acid when the residue is L and a negatively charged amino acid when the residue is K;

b) wherein the first canine IgG CH3 domain comprises an amino acid substitution at D383, D393 and/or S382 and the second canine IgG CH3 domain does not comprise a corresponding mutation or

c) or wherein the second canine IgG CH3 domain comprises an amino acid substitution at L463 or 425 and the first canine IgG CH3 domain does not comprise a corresponding mutation.

The IgG may be IgG-A, B, C or D. The above numbering refers to IgG-D, but a corresponding position in another isoform may be substituted.

In a further aspect, the invention relates to a heterodimeric protein comprising a first polypeptide comprising a first canine IgG CH3 domain and a second polypeptide comprising a second canine IgG CH3 domain and wherein
a) said first canine IgG CH3 domain comprises an amino acid substitution at one or more of position 428, 427, 382, 383 with an amino acid of the opposite charge and said second canine IgG CH3 domain comprises an amino acid substitution at one or more of position 420, 418, 437, 467, 396 with an amino acid of the opposite charge or
b) said second canine IgG CH3 domain comprises an amino acid substitution at 429 and the first canine IgG CH3 domain does not comprise a corresponding mutation.

For example, said first canine IgG CH3 domain comprises an amino acid substitution at one or more of position E428, D427, E382, E383, K386 with an amino acid of the opposite charge and said second canine IgG CH3 domain comprises an amino acid substitution at one or more of position R420, K418, K437, K467, K396, D397, D429 with an amino acid of the opposite charge.

The IgG may be IgG-A, B, C or D. The above numbering refers to IgG-B, but a corresponding position in another isoform may be substituted.

In a further aspect, the invention relates to a polypeptide comprising a canine IgG CH3 domain wherein said polypeptide comprises an amino acid substitution at one or more of the positions recited above (e.g. for IgG-B or IgG-D) with an amino acid of the opposite charge. In a further aspect, the invention relates to a nucleic acid encoding the heterodimeric protein or the polypeptide described herein.

In a further aspect, the invention relates to a vector comprising the nucleic acid or a host cell comprising the vector.

In a further aspect, the invention relates to a method for making a heterodimeric protein or a polypeptide with an amino acid substitution in a canine IgG CH3 domain comprising the steps of

a) transforming a host cell with a nucleic acid or a vector described herein;

b) culturing the host cell and expressing first and second IgG CH3 and

c) recovering the heterodimeric protein or polypeptide from the host cell culture.

In a further aspect, the invention relates to a pharmaceutical composition comprising the heterodimeric protein or polypeptide described herein and a pharmaceutical carrier.

In a further aspect, the invention relates to a kit comprising the heterodimeric protein or polypeptide described herein and optionally instructions for use.

### DESCRIPTION OF FIGURES

All mutation IDs mentioned below in the figure descriptions are with reference to IgG-B as shown in Table 2. The term combination/combination ID also refers to the mutation ID as shown in the table.

FIG. 1. KiH positions that are conserved in canine and human IgG isoforms.

FIG. 2. Residues forming CH3 charge pair interaction that are conserved between human and dog.

FIG. 3. Canine charge pair mutations enhance heterodimer formation.

FIG. 4. Heterodimerisation method evaluation of human IgG4PE KiH.

FIG. 5. Dog IgG-A, -B, -C, -D sequences (SEQ ID NO. 1, SEQ ID NO. 2, SEQ ID NO. 3, SEQ ID NO. 4 respectively).

FIG. 6. Alignment of Dog IgG-A, -B, -C, -D sequences (SEQ ID NO. 1, SEQ ID NO. 2, SEQ ID NO. 3, SEQ ID NO. 4). Highlighted residues are those described herein.

FIG. 7. Heterodimerisation evaluation (ratio 1:1) by SDS-PAGE, 1 of 3. Combinations/Mutation IDs 2 to 5 and 10 to 12 are expressed and form heterodimers. Mutation IDs 6 to 9 are not expressed. Region corresponding to Heterodimers MW is highlighted by dotted rectangle. Lanes are as follows: 1: molecular weight ladder (MW), 2: Combination 1, 3: Combination 2; 4: Combination 3; 5: Combination 4, 6: Combination 5, 7: Combination 6, 8: Combination 7, 9: Combination 8, 10: Combination 9, 11: Combination 10, 12: Combination 11, 13: Combination 12, 14: MW.

FIG. 8. Heterodimerisation evaluation (ratio 1:1) by SDS-PAGE, 2 of 3. Mutation IDs 13, 14 and 16 to 24 are expressed and form heterodimers. Mutation ID 15 is not expressed. Region corresponding to Heterodimers MW is highlighted by dotted rectangle. Lanes are as follows: 1: MW, 2: Combination 13, 3: Combination 14, 4: Combination 15, 5: Combination 16, 6: Combination 17, 7: Combination 18, 8: Combination 19, 9: Combination 20, 10: Combination 21, 11: Combination 22, 12: Combination 23, 13: Combination 24.

FIG. 9. Heterodimerisation evaluation (ratio 1:1) by SDS-PAGE, 3 of 3. Mutation IDs 25, 26 and 28 to 29 are expressed and form heterodimers. Mutation ID 27 is not expressed. Lanes are as follows: 1: Combination 25, 2: Combination 26, 3: Combination 27, 4: Combination 28, 5: Combination 29, 6: Sample protein, 7: MW.

FIG. 10. Densitometric analyses of Heterodimerisation evaluation (ratio 1:1) by SDS-PAGE. Values corresponding to Heterodimers are shown as % to total protein. Mutation ID 1 (WT) is shown as a comparison. The plot shows that Mutation IDs 3, 4, 10, 11, 12, 17, 19, 20, 21, 23 and 29 gave an increase in the proportion of heterodimers.

FIG. 11. Heterodimerisation evaluation (ratio 1:3) by SDS-PAGE, 1 of 2. All selected mutations, apart from Mutation ID 17, are expressed and form heterodimers. Region corresponding to Heterodimers MW is highlighted by dotted rectangle. Lanes are as follows: 1: MW, 2: Combination 1, 3: Combination 4, 4: Combination 11, 5: Combination 12, 6: Combination 13, 7: Combination 16, 8: Combination 17, 9: Combination 22, 10: Combination 23, 11: Combination 24, 12: Combination 25, 13: Combination 26, 14: Fc-wt Only, 15: scFv-wt only.

FIG. 12. Heterodimerisation evaluation (ratio 1:3) by SDS-PAGE, 2 of 2. All selected mutations are expressed and form heterodimers. The region of the gel corresponding to heterodimers MW is highlighted by dotted rectangle. Lanes are as follows: 1: MW, 2: Combination 1, 3: Combination 28, 4: Combination 29.

FIG. 13. Densitometric analyses of Heterodimerisation evaluation (ratio 1:3) by SDS-PAGE. Values corresponding to Heterodimers are shown as % to total protein. Mutation ID 1 (WT) is shown as comparison. The plot shows that for all combinations an increase in the proportion of heterodimers was observed apart from Mutation ID 17.

FIG. 14. Heterodimerisation evaluation (ratio 1:6) by SDS-PAGE, 1 of 2. All selected mutations are expressed and form heterodimers. Region corresponding to Heterodimers MW is highlighted by dotted rectangle. Lanes are as follows: 1: MW, 2: Combination 1, 3: Combination 4, 4: Combination 11, 5: Combination 12, 6: Combination 13, 7: Combination 16, 8: Combination 17, 9: Combination 22, 10: Combination 23, 11: Fc-wt Only, 12: scFv-wt only.

FIG. 15. Heterodimerisation evaluation (ratio 1:6) by SDS-PAGE, 2 of 2. All selected mutations are expressed and form heterodimers. Region corresponding to Heterodimers MW is highlighted by dotted rectangle. Lanes are as follows: 1: Combination 1, 2: MW, 3: Combination 24, 4: Combination 25, 5: Combination 26, 6: Combination 28, 7: Combination 29.

FIG. 16. Densitometric analyses of Heterodimerisation evaluation (ratio 1:6) by SDS-PAGE. Values corresponding to Heterodimers are shown as % to total protein. Mutation ID 1 (WT) is shown as comparison. The plot shows that all combinations increased Heterodimers proportion apart from ID 17 and 24.

FIG. 17a. HPLC SEC Retention time of both homodimers (highlighted with dotted lines) and heterodimers is shown. In all mutations a peak corresponding to heterodimers is detected and quantified.

FIG. 17b. shows -HPLC SEC quantification of Heterodimers peak shown as % of total protein.

FIG. 17c. shows HPLC SEC quantification of Heterodimers peak shown as total area (C).

FIG. 18. Table showing summary of results from 1:3 ratio. Combination/mutation ID 17 is highlighted in grey as giving the lowest expression of heterodimers.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols. 1 and 2, Ontermann and Duebel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The invention provides biological therapeutics for veterinary use, in particular for use in the treatment of dogs. In particular, the invention relates to heterodimeric, e.g. multispecific molecules, for targeting multiple disease-modifying molecules as well as methods and for producing such molecules. The invention is based on the manipulation of residues that form the interface of a dimeric protein, specifically residues in the canine IgG CH3 domain. The introduction of paired electrostatic steering amino acid mutations in the canine IgG CH3 domain creates altered charge polarity across the Fc dimer interface such that the heterologous heavy chains from different parental antibodies have strong and more specific interactions between each other, while the homodimer formation through homologous heavy chains is minimised due to repulsive charge achieved by the electrostatic steering mutations.

Therefore, in a first aspect, the invention relates to a heterodimeric protein comprising

a) a first polypeptide comprising a first canine IgG CH3 domain and

b) a second polypeptide comprising a second canine IgG CH3 domain

wherein said first and second canine IgG CH3 domain comprise one or more charge pair substitution.

The one or more charge pair substitution promotes heterodimerisation.

In a second aspect, the invention relates to a heterodimeric protein comprising

a) a first polypeptide comprising a first canine IgG CH3 domain and

b) a second polypeptide comprising a second canine IgG CH3 domain

wherein only one of the canine IgG CH3 domain comprise one or more charge pair substitution but the other does not.

The one or more charge pair substitution promotes heterodimerisation.

The term IgG CH3 domain refers to constant heavy chain 3 of an immunoglobulin (Ig) molecule.

The “first polypeptide” is any polypeptide that is to be associated with a second polypeptide, also referred to herein as “Chain A”. The first and second polypeptide meet/associate with each other at an “interface”. The “second polypeptide” is any polypeptide that is to be associated with the first polypeptide via an “interface”, also referred to herein as “Chain B”. The “interface” comprises those “contact” amino acid residues in the first polypeptide that interact with one or more “contact” amino acid residues in the interface of the second polypeptide. The canine IgG CH3 domain includes contact residues of such an interface.

As used herein, an interface comprises residues of a canine IgG CH3 domain, that is a CH3 domain of an Fc region that is derived from a canine or caninized IgG antibody.

A heterodimer or heterodimeric protein generally refers to a protein made up of two similar, but not identical subunits. An example of a heterodimeric protein is a bispecific antibody.

The term “antibody” as used herein refers to any immunoglobulin (Ig) molecule, or antigen binding portion or fragment thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. As used herein, the term “antibody” encompasses not only intact polyclonal or monoclonal antibodies.

In a full-length antibody, each heavy chain is comprised of a heavy chain variable region or domain (abbreviated herein as HCVR) and a heavy chain constant region. The heavy chain constant region is comprised of three constant heavy chain domains, CH1, CH2 and CH3. Each light chain is comprised of a light chain variable region or domain (abbreviated herein as LCVR) and a light chain constant region. The light chain constant region is comprised of one domain, CL.

Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies. The constant domains are not involved directly in the binding of antibody to an antigen, but exhibit various effector functions, such as participation of the antibody in antibody-dependent cellular toxicity.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can generally be of any isotype, class or subclass. The CH3 domain according to the various aspects of the invention is a CH3 domain of the canine IgG subtype, for example IgG-A, IgG-B, IgG-C, and IgG-D.

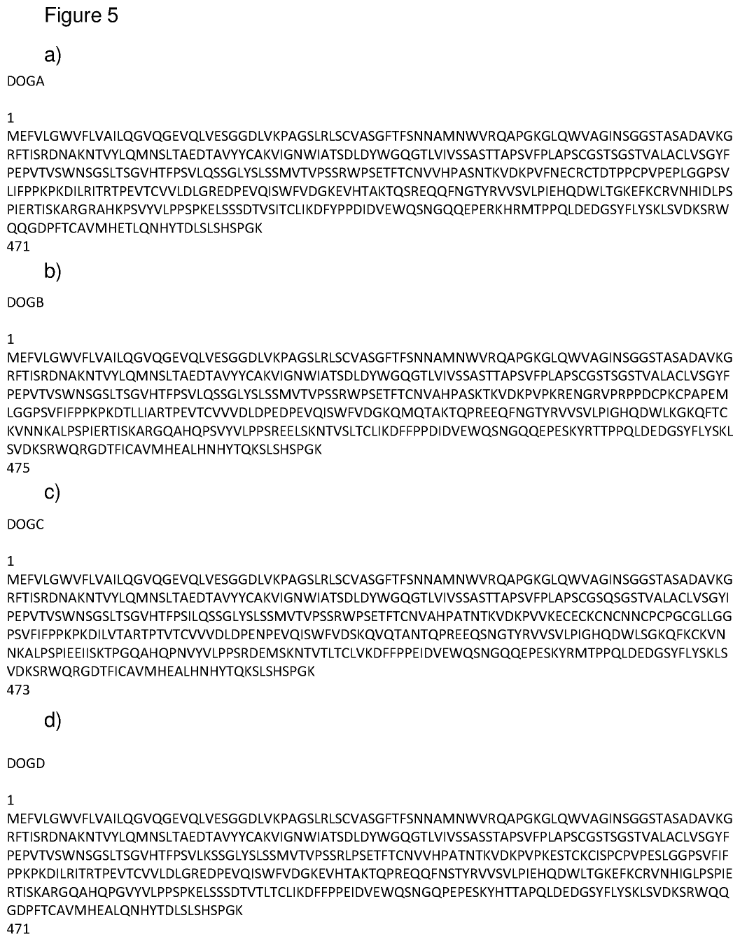

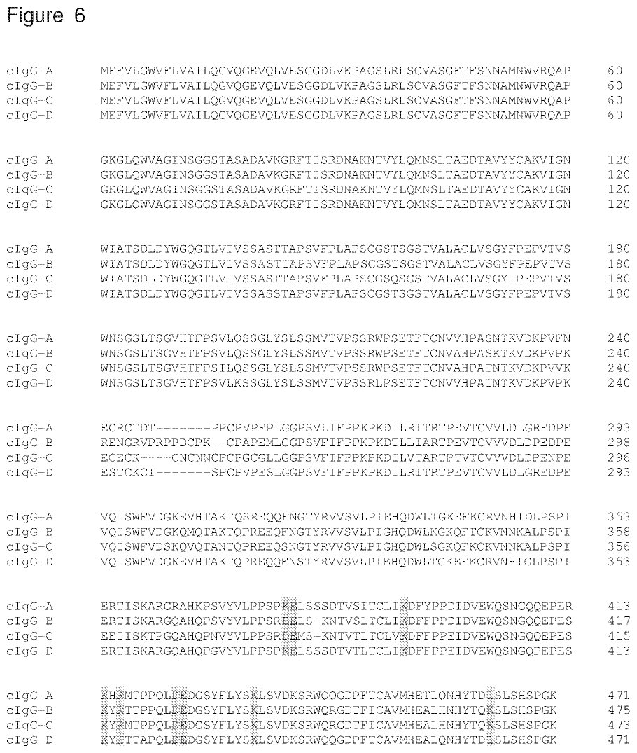

In canine, there are four IgG heavy chains referred to as A, B, C, and D. These heavy chains represent four different subclasses of dog IgG, which are referred to as IgG-A, IgG-B, IgG-C and IgG-D. The DNA and amino acid sequences of these four heavy chains were first identified by Tang et al. (Vet. Immunol. Immunopathol. 80: 259-270 (2001)). The amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Canine antibodies also contain two types of light chains, kappa and lambda (GenBank accession number kappa light chain amino acid sequence ABY 57289.1, GenBank accession number ABY 55569.1). Amino acid sequences are shown in FIGS. 5 and 6.

Thus, the invention specifically encompasses aspects that include a modification of the CH3 domain in canine IgG-A, IgG-B, IgG-C, or IgG-D. As explained above, residues at the defined positions may vary between isoforms. Also, the corresponding position in another isotope may be differently numbered as shown in FIG. 6.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are one of the most commonly used numbering system for human antibodies (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The terms “Kabat numbering”, “Kabat definitions” and “Kabat labelling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion.

The numbering used herein when referring to canine residues is with reference to canine IgG isoforms, specifically IgG-D and IgG-B as shown in FIG. 6. The first residue shown (M) is numbered 1 and the consecutive amino acid residues are given consecutive numbers. However, as mentioned above, the invention is not limited to these isoforms and the aspects of the invention also encompass the IgG-A, and

IgG-C isoforms. In these isoforms, the specific residue at the position for modification (e.g. 377, 416, 463) may be different to the residue found in IgG-D (see FIG. 6).

Proteolytic digestion of antibodies releases different fragments termed Fv (Fragment variable), Fab (Fragment antigen binding) and Fc (Fragment crystallisation). The Fc fragment comprises the carboxy-terminal portions of both H chains held together by disulfides. The constant domains of the Fc fragment are responsible for mediating the effector functions of an antibody.

Antigen binding fragments are also contemplated according to the aspects and embodiments of the invention. Antigen binding fragments include, for example, Fab, Fab′, F(ab′)2, Fd, Fv, single domain antibodies (sdAbs), e.g VH single domain antibodies, fragments including complementarity determining regions (CDRs), single chain variable fragment antibodies (scFv), maxibodies, minibodies, intrabodies, diabodies, triabodies, tetrabodies, and bis-scFv, and polypeptides that contain at least a portion of an immunoglobulin that is sufficient to confer specific antigen binding to the polypeptide.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site. “Single-chain Fv” also abbreviated as “sFv” or “scFv” are antibody fragments that comprise the VH and VL antibody domains connected into a single polypeptide chain.

The term “antigen binding site” refers to the part of the antibody or antibody fragment that comprises the area that specifically binds to an antigen. An antigen binding site may be provided by one or more antibody variable domains. Preferably, an antigen binding site is comprised within the associated VH and VL of an antibody or antibody fragment.

A “chimeric antibody” is a recombinant protein that contains the variable domains including the complementarity determining regions (CDRs) of an antibody derived from one species, while the constant domains of the antibody molecule are derived from those of another species, e.g. a canine antibody. An exemplary chimeric antibody is a chimeric human-canine antibody.

A “humanized antibody” is a recombinant protein in which the CDRs from an antibody from one species; e.g., a rodent antibody, are transferred from the heavy and light variable chains of the rodent antibody into human heavy and light variable domains (e.g., framework region sequences). The constant domains of the antibody molecule are derived from those of a human antibody. In certain embodiments, a limited number of framework region amino acid residues from the parent (rodent) antibody may be substituted into the human antibody framework region sequences.

As used herein, the term “caninized antibody” refers to forms of recombinant antibodies that contain sequences from both canine and non-canine (e.g., murine) antibodies. In general, the caninized antibody will comprise substantially all of at least one or more typically, two variable domains in which all or substantially all of the hypervariable loops correspond to those of a non-canine immunoglobulin, and all or substantially all of the framework (FR) regions (and typically all or substantially all of the remaining frame) are those of a canine immunoglobulin sequence. A caninized antibody may comprise both the three heavy chain CDRs and the three light chain CDRS from a murine or human antibody together with a canine frame or a modified canine frame. A modified canine frame comprises one or more amino acids changes that can further optimize the effectiveness of the caninized antibody, e.g., to increase its binding to its target.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins.

The term “epitope” or “antigenic determinant” refers to a site on the surface of an antigen (to which an immunoglobulin, antibody or antibody fragment, specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping) are well known in the art and include, for example, immunoblotting and immunoprecipitation assays, wherein overlapping or contiguous peptides from are tested for reactivity with a given antibody or antibody fragment. An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody.

The term “isolated” protein or polypeptide refers to a protein or polypeptide that is substantially free of other proteins or polypeptides, having different antigenic specificities. Moreover, protein or polypeptide may be substantially free of other cellular material and/or chemicals. Thus, the protein, nucleic acids and polypeptides described herein are preferably isolated. Thus, as used herein, an “isolated” protein, heterodimeric protein, heteromultimer or polypeptide means a heterodimeric protein, heteromultimer or polypeptide that has been identified and separated and/or recovered from a component of its natural cell culture environment. Contaminant components of its natural environment are materials that would interfere with diagnostic or therapeutic uses for the heterodimeric protein, heteromultimer or polypeptide, and may include enzymes, hormones, and other proteinaceous or non-proteinaceous solutes.

By “amino acid” herein is meant one of the 20 naturally occurring amino acids or any non-natural analogues that may be present at a specific, defined position. Amino acid encompasses both naturally occurring and synthetic amino acids. Although in most cases, when the protein is to be produced recombinantly, only naturally occurring amino acids are used.

As used herein, a “substitution of an amino acid residue” with another amino acid residue in an amino acid sequence of heterodimeric protein or polypeptide as described herein (an antibody for example), is equivalent to “replacing an amino acid residue” with another amino acid residue and denotes that a particular amino acid residue at a specific position in the original (e.g. wild type/germline) amino acid sequence has been replaced by (or substituted for) by a different amino acid residue. This can be done using standard techniques available to the skilled person, e.g. using recombinant DNA technology. The amino acids are changed relative to the native (wild type/germline) sequence as found in nature in the wild type (wt), but may be made in IgG molecules that contain other changes relative to the native sequence. By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified.

Thus, the invention provides variant CH3 domains which differ from the parent CH3 domain. By “parent polypeptide”, “parent protein”, “precursor polypeptide”, or “precursor protein” as used herein is meant an unmodified polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

In one embodiment of the heterodimeric protein, the first and/or second first canine IgG CH3 domain comprises one or more, e.g. 1, 2 or 3 amino acid substitutions.

By “position” herein is meant a location in the amino acid sequence of a protein, e.g. with reference to FIG. 5 or 6. Corresponding positions are determined as outlined, generally through alignment with other isoform sequences. The residue is the amino acid that occurs at a specified position.

For example, amino acids in the canine IgG CH3 are substituted such that amino acids of opposite side chain charges are introduced into the opposing CH3 domain. Thus, the one or more substitution replaces an amino acid with an amino acid of the opposite charge.

As mentioned above, the substitutions can be made in any canine IgG isoform and a skilled person would be able to identify a corresponding position in another isoform.

By “variant” or “mutant” herein is meant a polypeptide sequence that differs from that of a parent, e.g. wild-type sequence by virtue of at least one amino acid modification. As used herein, a “substitution of an amino acid residue” with another amino acid residue in an amino acid sequence of a protein or polypeptide as described herein, is equivalent to “replacing an amino acid residue” with another amino acid residue and denotes that a particular amino acid residue at a specific position in the original (e.g. wild type/germline) amino acid sequence has been replaced by (or substituted for) by a different amino acid residue. This can be done using standard techniques available to the skilled person, e.g. using recombinant DNA technology. The amino acids are changed relative to the native (wild type/germline) sequence as found in nature in the wild type (wt), but may be made in IgG molecules that contain other changes relative to the native sequence.

In certain embodiments, the heterodimeric protein comprises the specified amino acid substitutions and may have additional mutations in the CH3 domain or in another location in the amino acid sequence. In other embodiments, the heterodimeric protein only has the specified amino acid substitutions in the CH3 domain and does not have any other amino acid substitutions and/or other mutations in the CH3 domain. In other embodiments, the heterodimeric protein only has the specified amino acid substitutions in the CH3 domain and does not have any other mutations in the protein sequence. Thus, in one embodiment, the amino acid substitutions provided consist of those recited.

Modifications in IgG-D

The following sets out modifications with reference to the numbering of residues in the IgG-D amino acid sequence. This sequence is shown in FIGS. 5 and 6. The sequence of IgG-D is SEQ ID NO. 4. Thus, the numbering is with reference to SEQ ID NO. 4.

In another aspect, the invention relates to a heterodimeric protein comprising a first polypeptide comprising a first canine IgG CH3 domain and a second polypeptide comprising a second canine IgG CH3 domain wherein

a) said first canine IgG CH3 domain comprises an amino acid substitution at one or more of position 424, 423, 377, 378, 382 in IgG-D or at a corresponding position in IgG-A, B or D with an amino acid of the opposite charge and said second canine IgG CH3 domain comprises an amino acid substitution at one or more of position 414, 416, 433, 392, 377, 393 IgG-D or at a corresponding position in IgG-A, B or D with an amino acid of the opposite charge, an amino acid substitution at 463 with a charged amino acid or with an amino acid of the opposite charge and/or an amino acid substitution at 425 with N;

b) wherein the first canine IgG CH3 domain comprises an amino acid substitution at 383, 393 and/or 382 in IgG-D or at a corresponding position in IgG-A, B or D and the second canine IgG CH3 domain does not comprise a corresponding mutation or

c) wherein the second canine IgG CH3 domain comprises an amino acid substitution at 463 or 425 IgG-D or at a corresponding position in IgG-A, B or D and the first canine IgG CH3 domain does not comprise a corresponding mutation.

The invention relates to a heterodimeric protein wherein

a) said first canine IgG CH3 domain comprises an amino acid substitution at one or more of position E424, D423, D/K377, E378 IgG-D or at a corresponding position in IgG-A, B or D with an amino acid of the opposite charge and said second canine IgG CH3 domain comprises an amino acid substitution at one or more of position K414, H/R416, K433, K392 IgG-D or at a corresponding position in IgG-A, B or D with an amino acid of the opposite charge and/or an amino acid substitution at L/K463 with a charged amino acid when the residue is L and a negatively charged amino acid when the residue is K;

b) wherein the first canine IgG CH3 domain comprises an amino acid substitution at D383, D393 IgG-D or at a corresponding position in IgG-A, B or D and/or S382 IgG-D or at a corresponding position in IgG-A, B or D and the second canine IgG CH3 domain does not comprise a corresponding mutation or

c) or wherein the second canine IgG CH3 domain comprises an amino acid substitution at L463 or 425 IgG-D or at a corresponding position in IgG-A, B or D and the first canine IgG CH3 domain does not comprise a corresponding mutation.

In one embodiment, with reference to the specific residues in canine IgG-D, said first canine IgG CH3 domain comprises an amino acid substitution at one or more of position E424, D423, K377 or E378 with an amino acid of the opposite charge and said second canine IgG CH3 domain comprises an amino acid substitution at one or more of position K414, H416, K433, K392 with an amino acid of the opposite charge and/or an amino acid substitution at L463 with a charged amino acid or wherein the second canine IgG CH3 domain comprises an amino acid substitution at L463 and the first canine IgG CH3 domain does not comprise a corresponding mutation. The skilled person will appreciate that in other canine IgG isoforms, a different residue can be found at the specific positions. This is shown in FIG. 6. For example, in canine IgG-C, at position 377, the residue is D. In canine isoforms IgG-A, IgG-B and IgG-C, the residue at position 416 is R.

The substitutions with reference to the specific residues in canine IgG-D may be selected as follows: the substitution at E424 is E424K, E424R or E424H, the substitution at D423 is D423K, D423R or D423H, the substitution at E378 is E378K, E378R or E378H, the substitution at K377 is K377E or K377D, the substitution at D377 is D377K, D377R or D377H, the substitution at K414 is K414E or K414D, the substitution at H/R416 is H/R416D or H/R416E, the substitution at K433 is K433D or K433E, the substitution at L463 is L463K, L463R, L463H L463E or L463D, the substitution at K463 is K463D or K463E and the substitution at K392 is K392E or K392D.

In one embodiment, the first canine IgG CH3 domain comprises an amino acid substitution at position E424 with a positively charged amino acid and said second canine IgG CH3 domain comprises an amino acid substitution at position K414 with a negatively charged amino acid.

In one embodiment, the first canine IgG CH3 domain comprises an amino acid substitution at position E424 with a positively charged amino acid and said second canine IgG CH3 domain comprises an amino acid substitution at position K414 with a negatively charged amino acid and an amino acid substitution at position H/R416 with a negatively charged amino acid. In one embodiment, the substitution at E424 is E424K, the substitution at K414 is K414E and the substitution at H/R416 is H/R416D.

In one embodiment, the first canine IgG CH3 domain comprises an amino acid substitution at position D423 with a positively charged amino acid and the second canine IgG CH3 domain comprises an amino acid substitution at position K433 with a negatively charged amino acid. In one embodiment, the substitution at D423 is D423K, the substitution at K433 is K433D.

In one embodiment, the first canine IgG CH3 domain comprises an amino acid substitution at position D423 with a positively charged amino acid and said second canine IgG CH3 domain comprises an amino acid substitution at position K433 with a negatively charged amino acid and an amino acid substitution at position H/R416 with a negatively charged amino acid. In one embodiment, the substitution at D423 is D423K, the substitution at K433 is K433D and the substitution at H/R416 is H/R416D.

In one embodiment, the first canine IgG CH3 domain comprises an amino acid substitution at position D423 with a positively charged amino acid amino and an acid substitution at position E424 with a positively charged amino acid and the second canine IgG CH3 domain comprises an amino acid substitution at position K433 with a negatively charged amino acid and an amino acid substitution at position K414 with a negatively charged amino acid. In one embodiment, the substitution at E424 is E424K, the substitution at K414 is K414E and the substitution at K433 is K433D.

In one embodiment, the first canine IgG CH3 domain comprises an amino acid substitution at position D423 with a positively charged amino acid amino and an acid substitution at position E424 with a positively charged amino acid and the second canine IgG CH3 domain comprises an amino acid substitution at position K433 with a negatively charged amino acid, an amino acid substitution at position K414 with a negatively charged amino acid and an amino acid substitution at position H/R416 with a negatively charged amino acid. In one embodiment, the substitution at E424 is E424K, the substitution at K414 is K414E and the substitution at H/R416 is H/R416D.

In one embodiment, the second canine IgG CH3 domain comprises an amino acid substitution at position L463 with a positively charged amino acid and the first canine IgG CH3 domain does not comprise a corresponding mutation.

In one embodiment, the first canine IgG CH3 domain comprises an amino acid substitution at position E378 with a positively charged amino acid and said second canine IgG CH3 domain comprises an amino acid substitution at position at position K392 with a negatively charged amino acid. In one embodiment, the substitution at E378 is E378K, the substitution at K392 is K392E.

In one embodiment, the first canine IgG CH3 domain comprises an amino acid substitution at position E424 with a positively charged amino acid and said second canine IgG CH3 domain comprises an amino acid substitution at position at position K414 with a negatively charged amino acid, an amino acid substitution at position H/R416 with a negatively charged amino acid and an amino acid substitution at position L/K463 with a charged amino acid when the residue is L and a negatively charged amino acid when the residue is K. In one embodiment, the substitution at E424 is E424K, the substitution at K414 is K414E, the substitution at H/R416 is H/R416D and the substitution at L463 is L463K.

In one embodiment, the first canine IgG CH3 domain comprises an amino acid substitution at position D423 with a positively charged amino acid and said second canine IgG CH3 domain comprises an amino acid substitution at position at position K433 with a negatively charged amino acid, an amino acid substitution at position H/R416 with a negatively charged amino acid and an amino acid substitution at position L/K463 with a charged amino acid when the residue is L and a negatively charged amino acid when the residue is K. In one embodiment, the substitution at D423 is D423K, the substitution at K333 is K433E, the substitution at H/R416 is H/R416D and the substitution at L463 is L463K.

In one embodiment, the first canine IgG CH3 domain comprises an amino acid substitution at position E424 with a positively charged amino acid and the second canine IgG CH3 domain comprises an amino acid substitution at position at position K414 with a negatively charged amino acid, and an amino acid substitution at position L463 with a positively charged amino acid. In one embodiment, the substitution at E424 is E424K, the substitution at K414 is K414E and the substitution at L463 is L463E.

In one embodiment, the first canine IgG CH3 domain comprises an amino acid substitution at position D423 with a positively charged amino acid and the second canine IgG CH3 domain comprises an amino acid substitution at position at position K433 with a negatively charged amino acid, and an amino acid substitution at position L/K463 with a charged amino acid when the residue is L and a negatively charged amino acid when the residue is K. In one embodiment, the substitution at D423 is D423K, the substitution at K333E is K433E, and the substitution at L463 is L463K.

In one embodiment, the first canine IgG CH3 domain comprises an amino acid substitution at position E424 with a positively charged amino acid and an amino acid substitution at position D423 with a positively charged amino acid and said second canine IgG CH3 domain comprises an amino acid substitution at position at position K414 with a negatively charged amino acid, an amino acid substitution at position K433 with a negatively charged amino acid and an amino acid substitution at position L/K463 with a charged amino acid when the residue is L and a negatively charged amino acid when the residue is K. In one embodiment, the substitution at E424 is E424K, the substitution at D423 is D423K, the substitution at K414 is K414E and the substitution at L463 is L463K.

In one embodiment, the first canine IgG CH3 domain comprises an amino acid substitution at position E424 with a positively charged amino acid and an amino acid substitution at position K/D377 with a negatively charged amino acid if the residue is K or a positively charged amino acid if the residue is D and said second canine IgG CH3 domain comprises an amino acid substitution at position at position K414 with a negatively charged amino acid, and an amino acid substitution at position L/K463 with a charged amino acid when the residue is L and a negatively charged amino acid when the residue is K. In one embodiment, the substitution at E424 is E424K, the substitution at K377 is K377E, the substitution at K414 is K414E and the substitution at L463 is L463K.

In one embodiment, the first canine IgG CH3 domain comprises an amino acid substitution at position D423 with a positively charged amino acid and an amino acid substitution at position K/D377 with a negatively charged amino acid if the residue is K or a positively charged amino acid if the residue is D and said second canine IgG CH3 domain comprises an amino acid substitution at position at position K433 with a negatively charged amino acid, and an amino acid substitution at position L/K463 with a charged amino acid when the residue is L and a negatively charged amino acid when the residue is K. In one embodiment, the substitution at D423 is D423K, the substitution at K/D377 is K/D377E, the substitution at K333E is K433E and the substitution at L463 is L463K.

In one embodiment, the first canine IgG CH3 domain comprises an amino acid substitution at position E424 with a positively charged amino acid, an amino acid substitution at position D423 with a positively charged amino acid and an amino acid substitution at position K/D377 with a negatively charged amino acid if the residue is K or a positively charged amino acid if the residue is D and said second canine IgG CH3 domain comprises an amino acid substitution at position at position K414 with a negatively charged amino acid, an amino acid substitution at position at position K433 with a negatively charged amino acid and an amino acid substitution at position L/K463 with a charged amino acid when the residue is L and a negatively charged amino acid when the residue is K. In one embodiment, the substitution at D423 is D423K, the substitution at E424 is E424K, the substitution at K377 is K377E, the substitution at K414 is K414E and the substitution at K433 is K433D and the substitution at L463 is L463K.

In one embodiment, the first canine IgG CH3 domain comprises an amino acid substitution at position E378 with a positively charged amino acid, and said second canine IgG CH3 domain comprises an amino acid substitution at position K392 with a negatively charged amino acid. In one embodiment, the amino acid substitution at position E378 is E378K, and the amino acid substitution at position K392 is K392E.

In one embodiment, the first canine IgG CH3 domain comprises an amino acid substitution at position D393 with a positively charged amino acid, and said second canine IgG CH3 domain comprises an amino acid substitution at position K377 with a negatively charged amino acid. In one embodiment, the amino acid substitution at position D393 is D393K and the amino acid substitution at position K377 is K377D.

In one embodiment, the first canine IgG CH3 domain comprises an amino acid substitution at position E378 with a positively charged amino acid and an amino acid substitution at position D393 with a positively charged amino acid, and said second canine IgG CH3 domain comprises an amino acid substitution at position K392 with a negatively charged amino acid and an amino acid substitution at position K377 with a negatively charged amino acid. In one embodiment, the amino acid substitution at position E378 is E378K, the amino acid substitution at position K392 is K392E, the amino acid substitution at position D393 is D393K and the amino acid substitution at position K377 is K377D.

In one embodiment, the first canine IgG CH3 domain comprises an amino acid substitution at position K377 with a negatively charged amino acid, and said second canine IgG CH3 domain comprises an amino acid substitution at position L/K463 with a charged amino acid when the residue is L and a negatively charged amino acid when the residue is K. In one embodiment, the amino acid substitution at position K377 is K377E and amino acid substitution at position L463 is L463K.

In one embodiment, the first canine IgG CH3 domain comprises an amino acid substitution at position S382 with a positively charged amino acid and said second canine IgG CH3 domain does not comprise a mutation. In one embodiment, the amino acid substitution at position S382 is S382K.

In one embodiment, the first canine IgG CH3 domain comprises an amino acid substitution at position D383 with a negatively charged amino acid and said second canine IgG CH3 domain does not comprise a mutation. In one embodiment, the amino acid substitution at position D383 is D383N.

In one embodiment, the amino acid substitution at position first canine IgG CH3 domain comprises an amino acid substitution at position S382 with a positively charged amino acid and an amino acid substitution at position D393 with N and said second canine IgG CH3 domain does not comprise a mutation. In one embodiment, the amino acid substitution at position S382 is S382K and the amino acid substitution at position D393 is D393N.

In one embodiment, the first canine IgG CH3 domain comprises an amino acid substitution at position S382 with a negatively charged amino acid and said second canine IgG CH3 comprises an amino acid substitution at position D393 with a negatively charged amino acid. In one embodiment, the amino acid substitution at position S382 is S382D and the amino acid substitution at position D393 is D393K.

In one embodiment, the first canine IgG CH3 domain does not comprise an amino acid substitution and said second canine IgG CH3 comprises an amino acid substitution at position D425 with N.

In one embodiment, the first canine IgG CH3 domain comprises an amino acid substitution at position S382 with a negatively charged amino acid, and said second canine IgG CH3 domain comprises an amino acid substitution at position D393 with a negatively charged amino acid and at D425 with N. In one embodiment, the amino acid substitution at position S382 is S383D. In one embodiment, the amino acid substitution at position D393 is D393K and the amino acid substitution at position D383 is D383N.

Thus, in one embodiment, the substitution at E424 is E424K, E424R or E424H, the substitution at D423 is D423K, D423R or D423H, the substitution at E378 is E378K, E378R or E378H, the substitution at K377 is K377E or K377D, the substitution at D377 is D377K, D377R or D377H, the substitution at K414 is K414E or K414D, the substitution at H/R416 is H/R416D or H/R416E, the substitution at K433 is K433D or K433E, the substitution at L463 is L463K, L463R, L463H L463E or L463D, the substitution at K463 is K463D or K463E, the substitution at K392 is K392E or K392D, the substitution at E378 is E378K, E378R or E378H, the substitution at D393 is D393K, D393R or D393H, the substitution at S382 is S382K, S382R or S382H, the substitution at D383 is D383N the substitution at D425 is N.

In one embodiment, the mutation in the CH3 domain is selected from one of mutation IDs 1 to 26 as shown in table 1. Mutations in mutation ID 27 may be additionally included.

Also within the scope of the invention is a modified CH3 domain e.g. an isolated CH3 domain, that is a CH3 domain which has an amino acid substitution at one or more of position 428, 427, 382, 383, 386, 420, 418, 437, 467, 396, 397, 429, 424, 423, 377, 378, 382, 414, 416, 433, 392, 377, 393, 463, 425, 383, 393 and/or 382. The substitution may be with an amino acid of an opposite charge and can be selected from those shown in Table 1.

As mentioned above, the substitutions can be made in any canine IgG isoform and a skilled person would be able to identify a corresponding position in another isoform.

Modifications in IgG-B

The following sets out modifications with reference to the numbering of residues in the IgG-B amino acid sequence. This sequence is shown in FIGS. 5 and 6. The sequence of IgG-B is SEQ ID NO. 2. Thus, the numbering is with reference to SEQ ID NO. 2.

In one aspect, the invention relates to a heterodimeric protein comprising a first polypeptide comprising a first canine IgG CH3 domain and a second polypeptide comprising a second canine IgG CH3 domain wherein said IgG is selected from IgG-A, B, C or D and wherein

a) said first canine IgG CH3 domain comprises an amino acid substitution at one or more of position 428, 427, 382, 383 in IgG-B or at a corresponding position in IgG-A, C or D with an amino acid of the opposite charge and said second canine IgG CH3 domain comprises an amino acid substitution at one or more of position 420, 418, 437, 467, 396 in IgG-B or at a corresponding position in IgG-A, C or D with an amino acid of the opposite charge

b) said second canine IgG CH3 domain comprises an amino acid substitution at 429 in IgG-B or at a corresponding position in IgG-A, C or D and the first canine IgG CH3 domain does not comprise a corresponding mutation or

c) said first canine IgG CH3 domain comprises an amino acid substitution at 387 in IgG-B or at a corresponding position in IgG-A, C or D and the second IgG CH3 domain does not comprise a corresponding mutation.

For example, said first canine IgG CH3 domain comprises an amino acid substitution at one or more of position E428, D427, E382, E383, with an amino acid of the opposite charge and said second canine IgG CH3 domain comprises an amino acid substitution at one or more of position R420, K418, K437, K467, K396 with an amino acid of the opposite charge.

In one embodiment, said first canine IgG CH3 domain comprises an amino acid substitution at position E428 with a positively charged amino acid and said second canine IgG CH3 domain comprises an amino acid substitution at position R420 with a negatively charged amino acid.

In one embodiment, said first canine IgG CH3 domain comprises an amino acid substitution at position E428 with a positively charged amino acid and said second canine IgG CH3 domain comprises an amino acid substitution at position R420 with a negatively charged amino acid and an amino acid substitution at position K418 with a negatively charged amino acid.

In one embodiment, said first canine IgG CH3 domain comprises an amino acid substitution at position D427 with a positively charged amino acid and said second canine IgG CH3 domain comprises an amino acid substitution at position R420 with a negatively charged amino acid.

In one embodiment, said first canine IgG CH3 domain comprises an amino acid substitution at position D427 with a positively charged amino acid and said second canine IgG CH3 domain comprises an amino acid substitution at position R420 with a negatively charged amino acid and an amino acid substitution at position K437 with a negatively charged amino acid.

In one embodiment, said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position K437 with a negatively charged amino acid and an amino acid substitution at position K418 with a negatively charged amino acid.

In one embodiment, said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position R420 with a negatively charged amino acid.

In one embodiment, said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position R420 with a negatively charged amino acid and an amino acid substitution at position K437 with a negatively charged amino acid.

In one embodiment, said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position K420 with a negatively charged amino acid and an amino acid substitution at position K418 with a negatively charged amino acid.

In one embodiment, said first canine IgG CH3 domain comprises an amino acid substitution at position E382 with a positively charged amino acid amino acid and said second canine IgG CH3 domain comprises an amino acid substitution at position K467 with a negatively charged amino acid.

In one embodiment, said first canine IgG CH3 domain comprises an amino acid substitution at position E383 with a positively charged amino acid amino acid and an amino acid substitution at position K396 with a negatively charged amino acid.

In one embodiment, said first canine IgG CH3 domain comprises an amino acid substitution at position E382 with a positively charged amino acid and an amino acid substitution at position D427 with a positively charged amino acid and said second canine IgG CH3 domain comprises an amino acid substitution at position K418 with a negatively charged amino acid and at position R420 with a negatively charged amino acid.

In one embodiment, said first canine IgG CH3 domain comprises an amino acid substitution at position E382 with a positively charged amino acid and an amino acid substitution at position E428 with a positively charged amino acid and said second canine IgG CH3 domain comprises an amino acid substitution at position K418 with a negatively charged amino acid and at position R420 with a negatively charged amino acid.

In one embodiment, said first canine IgG CH3 domain comprises an amino acid substitution at position E382 with a positively charged amino acid and an amino acid substitution at position D427 with a positively charged amino acid and said second canine IgG CH3 domain comprises an amino acid substitution at position K437 with a negatively charged amino acid and at position R420 with a negatively charged amino acid.

In one embodiment, said first canine IgG CH3 domain comprises an amino acid substitution at position E382 with a positively charged amino acid and an amino acid substitution at position E428 with a positively charged amino acid and said second canine IgG CH3 domain comprises an amino acid substitution at position K437 with a negatively charged amino acid and at position R420 with a negatively charged amino acid.

In one embodiment, said first canine IgG CH3 domain comprises an amino acid substitution at position E383 with a positively charged amino acid and an amino acid substitution at position D427 with a positively charged amino acid and said second canine IgG CH3 domain comprises an amino acid substitution at position K418 with a negatively charged amino acid and at position R420 with a negatively charged amino acid.

In one embodiment, said first canine IgG CH3 domain comprises an amino acid substitution at position E383 with a positively charged amino acid and an amino acid substitution at position E428 with a positively charged amino acid and said second canine IgG CH3 domain comprises an amino acid substitution at position K418 with a negatively charged amino acid and at position R420 with a negatively charged amino acid.

In one embodiment, said first canine IgG CH3 domain comprises an amino acid substitution at position E383 with a positively charged amino acid and an amino acid substitution at position D427 with a positively charged amino acid and said second canine IgG CH3 domain comprises an amino acid substitution at position K437 with a negatively charged amino acid and at position R420 with a negatively charged amino acid.

In one embodiment, said first canine IgG CH3 domain comprises an amino acid substitution at position E383 with a positively charged amino acid and an amino acid substitution at position E428 with a positively charged amino acid and said second canine IgG CH3 domain comprises an amino acid substitution at position K437 with a negatively charged amino acid and at position R420 with a negatively charged amino acid.

In one embodiment, wherein said first canine IgG CH3 domain comprises an amino acid substitution at position E428 with a positively charged amino acid and said second canine IgG CH3 domain comprises an amino acid substitution at position K418 with a negatively charged amino acid and at position K467 with a negatively charged amino acid.

In one embodiment, said first canine IgG CH3 domain comprises an amino acid substitution at position D427 with a positively charged amino acid and said second canine IgG CH3 domain comprises an amino acid substitution at position K437 with a negatively charged amino acid and at position R467 with a negatively charged amino acid.

In one embodiment, said first canine IgG CH3 domain comprises an amino acid substitution at position E428 with a positively charged amino acid and said second canine IgG CH3 domain comprises an amino acid substitution at position K418 with a negatively charged amino acid and at position K396 with a negatively charged amino acid.

In one embodiment, said first canine IgG CH3 domain comprises an amino acid substitution at position D427 with a positively charged amino acid and said second canine IgG CH3 domain comprises an amino acid substitution at position K437 with a negatively charged amino acid and at position K396 with a negatively charged amino acid.

In one embodiment, said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position R420 with a negatively charged amino acid and at position K467 with a negatively charged amino acid.

In one embodiment, said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position K396 with a negatively charged amino acid and at position K467 with a negatively charged amino acid.

In one embodiment, said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position K437 with a negatively charged amino acid, at position K418 with a negatively charged amino acid and at position K467 with a negatively charged amino acid.

In one embodiment, said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position K437 with a negatively charged amino acid, at position K418 with a negatively charged amino acid and at position K396 with a negatively charged amino acid.

In one embodiment, wherein said first canine IgG CH3 domain comprises an amino acid substitution at position E428 with a positively charged amino acid and said second canine IgG CH3 domain comprises an amino acid substitution at position K418 with a negatively charged amino acid.

In one embodiment, said first canine IgG CH3 domain comprises an amino acid substitution at position D427 with a positively charged amino acid and said second canine IgG CH3 domain comprises an amino acid substitution at position K437 with a negatively charged amino acid.

In one embodiment, said first canine IgG CH3 domain comprises an amino acid substitution at position K386 with a negatively charged amino acid and said second canine IgG CH3 domain comprises an amino acid substitution at position D397 with a positively charged amino acid.

In one embodiment, said first canine IgG CH3 domain does not comprises an amino acid substitution and said second canine IgG CH3 domain comprises an amino acid substitution at position D429.

In one embodiment, said first canine IgG CH3 domain comprises an amino acid substitution at position K386 with a negatively charged amino acid and said second canine IgG CH3 domain comprises an amino acid substitution at position D397 with a positively charged amino acid and a substitution at D429.

In one embodiment, said first canine IgG CH3 domain comprises an amino acid substitution at position N387 with a negatively charged amino acid and said second canine IgG CH3 domain does not comprise an amino acid substitution.

In one embodiment, the substitution at E428 is E428K, E428R or E428H, the substitution at D427 is D427K, D427R or D427H, the substitution at E382 is E382K, E382R or E382H, the substitution at E383 is E383K, E383R, E383H or E383R, the substitution at R420 is R420E or R420D, the substitution at K418 is K418E or K418D, the substitution at K437 is K437D or K437E, the substitution at K467 is K467D or K467E, the substitution at K396 is K396E or K396D, the substitution at K386 is K386E or K386D, the substitution at D397 is D397K, D397R or D397H and the substitution at D429 is D429N or the substitution at N387 is N387D.

In one embodiment, the substitution at E428 is E428K, the substitution at D427 is D427K, the substitution at E382 is E382K, the substitution at E383 is E383K, the substitution at R420 is R420D, the substitution at K418 is K418E, the substitution at K437 is K437D, the substitution at K467 is K467E, the substitution at K396 is K396E, the substitution at K386 is K386D, the substitution at D397 is D397K and the substitution at D429 is D429N.

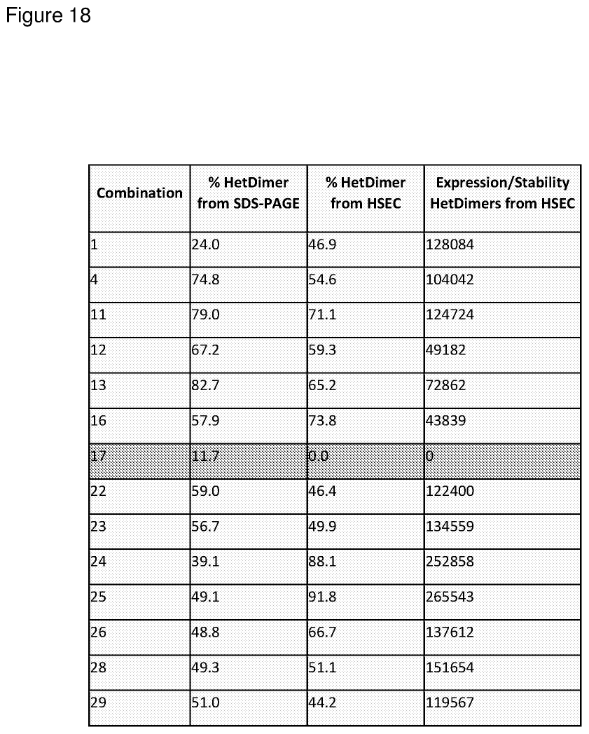

In one embodiment, the mutation in the CH3 domain is selected from one of mutation IDs as shown in table 2. In one embodiment, the mutation in the CH3 domain is selected from one of the following mutation IDs as shown in table 2: 2, 3, 4, 5, 10, 11, 12, 13, 14, 16, 17, 18, 19, 20, 21, 22, 23, 24, 25, 26, 28 or 29. In one embodiment, the mutation in the CH3 domain is selected from one of combination IDs as shown in FIG. 18. In one embodiment, the mutation in the CH3 domain is selected from one of combination IDs 24 and 25.

Also within the scope of the invention is a modified CH3 domain, e.g. an isolated CH3 domain, that is a CH3 domain which has an amino acid substitution at one or more of position 428, 427, 382, 383, 386, 420, 418, 437, 467, 396, 397, 429. The substitution may be with an amino acid of an opposite charge and can be selected from one of the substitutions as shown in Table 2.

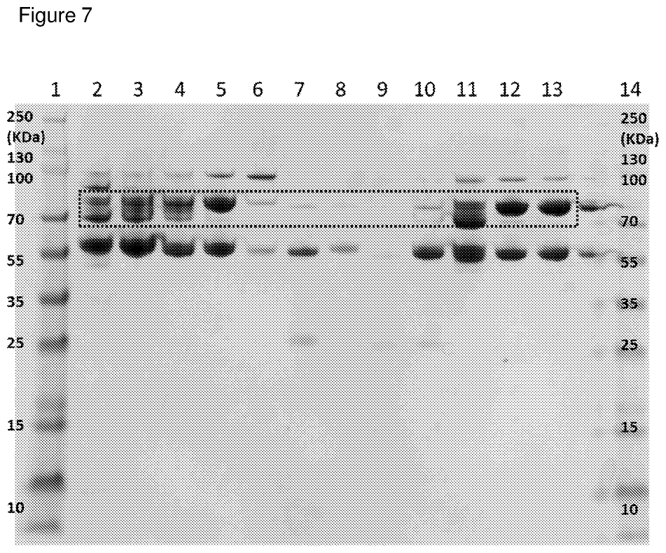

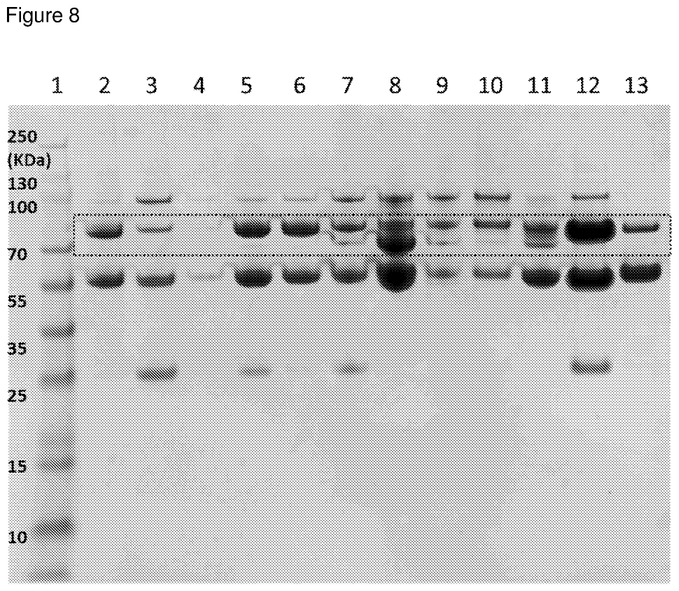

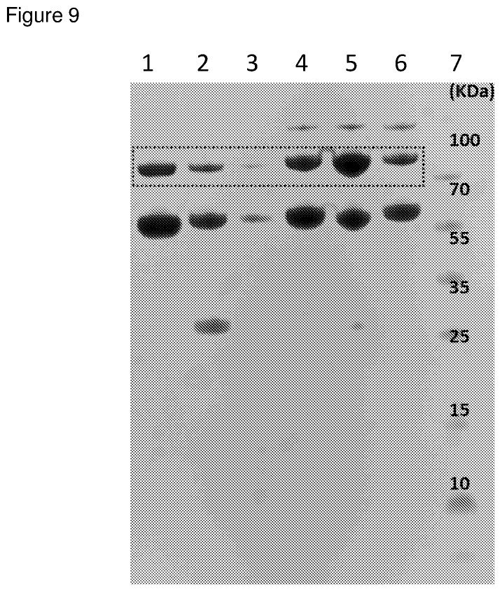

Challenges in bispecific antibodies include problems for assembly and purification, production challenges (heavy chain heterodimerisation, light chain pairing, bispecific purification over monospecific contaminants). As shown in the examples and FIGS. 7, 8 and 9, under the experimental conditions used, certain combinations of mutations generate molecules which do not express, or which do not pair, or which form a “blur” on the gel i.e. multiple possible formats rather than clear bispecific molecule being generated. Therefore, in some embodiments, the aspects of the invention relate to mutation IDs 2-5, 10-14, 16-26, 28 and 29 with reference to Table 2.

In one embodiment, a heterodimeric protein as described herein may include additional mutations in the CH3 domain or may not include additional mutations in the CH3 domain. For example, 1, 2, 3, 4, 5 or more additional mutations may be included in the CH3 domain. The additional mutations may be mutations that promote heterodimerisation. Therefore, a skilled person will understand that any of the combinations set out above can be further combined with such additional mutations in the CH3 domain.

In one example, the one or more additional mutations are introduced to reproduce the “knob in hole Fc technology” (KiH). Using this technique, a larger amino acid tyrosine is introduced to take the place of a small one in the CH3 domain of a first polypeptide, forming the “knob”. The second polypeptide is manipulated in the opposite way, substitution of larger amino acids with smaller ones to generate the “hole”. This creates a steric hindrance effect which promotes the desired heterodimeric assembly between a first and a second polypeptide having a canine IgG CH3 domain.

For example, the first canine IgG CH3 domain comprises an amino acid substitution at position T388 and said second canine IgG CH3 domain comprises an amino acid substitution at position T388, L390 and Y431 with reference to IgG-D numbering.

In one embodiment, the mutation is a substitution at one or more of positions T388, L390 and Y431 and is selected from T388W, T388S, L390A and Y431V with reference to IgG-D numbering. With reference to IgG-B numbering, these are T392S, L394A, Y435V and T392W.

The skilled person will appreciate that different mutations in the CH3 domain e.g. those that alter the charge of the Fc domain interface and those that use “knob in hole Fc technology” (KiH) can be combined in the molecules and methods of the invention.

The amount of homodimer formation is reduced by at least 10%, 20%, 30%, 40%, 50%, 60%, 70%, 80%, 90%, 95%, 96%, 97%, 98% or 99% when using a modified polypeptide of the invention, for example as determined by mass spectrometry.

The variants of the invention include one or more substitution in the CH3 domain and they can include any number of further modifications, as long as the function of the protein is still present, as described herein. However, in general, from 1, 2, 3, 4, 5, 6, 7, 8, 9 or 10 modifications in addition to the CH3 modifications are generally utilized as often the goal is to alter function with a minimal number of modifications. In some cases, there are from 1 to 5 modifications, with from 1-2, 1-3 and 1-4 also finding use in many embodiments. It should be noted that the number of amino acid modifications may be within functional domains: for example, it may be desirable to have from 1-6 modifications in the Fc region of wild-type or engineered proteins, as well as from 1 to 6 modifications in the CH3 region, for example. A variant polypeptide sequence will preferably possess at least about 80%, 85%, 90%, 95% or up to 98% or 99% identity to the wild-type sequences or the parent sequences. It should be noted that depending on the size of the sequence, the percent identity will depend on the number of amino acids.

Among the many platforms for creating bsAbs, controlled Fab-arm exchange (cFAE) has proven useful based on minimal changes to native Ab structure and the simplicity with which bsAbs can be formed from two parental Abs (Labrijn et al, (2013) Efficient generation of stable bispecific IgG1 by controlled Fab-arm exchange. Proc. Natl. Acad. Sci. U.S.A. 110, 5145-5150). Controlled Fab-arm exchange uses a minimal set of mutations and avoids the light chain pairing issue, as exchange of half-Abs can be performed without perturbing the correct heavy chain-light chain interaction. Thus, cFAE can also be used in the present invention in addition to mutations set out above.

The present invention thus also provides modified canine IgGs which comprise a hinge region from another isoform in place of its natural IgG hinge region, e.g. IgG-Ds which comprise a hinge region from either IgG-A, IgG-B, or IgG-C in place of its natural IgG-D hinge region or IgG-B. which comprise a hinge region from either IgG-A, IgG-C, or IgG-D in place of its natural IgG-B hinge region Such modifications can lead to a canine IgG-D lacking fab arm exchange. The modified canine IgG can be constructed using standard methods of recombinant DNA technology [e.g., Maniatis et al., Molecular Cloning, A Laboratory Manual (1982)]. In order to construct these variants, the nucleic acids encoding the amino acid sequence of canine IgG, for example IgG-B or IgG-D can be modified so that it encodes the modified IgG. The modified nucleic acid sequences are then cloned into expression plasmids for protein expression

In one embodiment of the heterodimeric protein of the invention, the heterodimeric protein comprises a Fc region, e.g. a canine or Fc region or one that is derived from a canine Fc region.

In one embodiment of the heterodimeric protein of the invention, the first polypeptide is an antibody heavy chain and/or said second polypeptide is an antibody heavy chain, e.g. a canine antibody heavy chain, caninized antibody heavy chain or one that is derived from a canine antibody heavy chain. In one embodiment, both the first polypeptide and said second polypeptide are antibody heavy chains, e.g. a canine antibody heavy chains, caninized antibody heavy chains or Fc region or one that is derived from a canine antibody heavy chain.

In one embodiment of the heterodimeric protein of the invention, the heterodimeric protein comprises a first and a second light chain, e.g. a canine antibody light chain, caninized antibody light chain or one that is derived from a canine antibody light chain.

In one embodiment, the heterodimeric protein is an antibody, e.g. a canine antibody, caninized antibody light chain or one that is derived from a canine antibody.

In one embodiment, the antibody is a multispecific antibody or fragment thereof. A multispecific protein, e.g. a multispecific antibody, binds to at least two different targets, i.e. is at least bispecific. Thus, in one embodiment, the antibody is a bispecific antibody or fragment thereof. In other embodiment, the mutispecific antibody or fragment thereof binds to three, four or more targets.

In one embodiment, in particular for the treatment of cancer, the protein may target CD3 and is provided in the format of a bispecific T-cell engager (BiTE).

In one embodiment, the protein is multiparatopic, i.e. binds to more than one epitope on the same target.

A bispecific antibody has specificity for no more than two epitopes. A bispecific antibody is characterized by a first immunoglobulin variable domain sequence which has binding specificity for a first epitope and a second immunoglobulin variable domain sequence that has binding specificity for a second epitope. In one embodiment, the first and second epitopes are on the same antigen, e.g., the same protein (or subunit of a multimeric protein). In one embodiment, the first and second epitopes overlap. In an embodiment, the first and second epitopes do not overlap. In one embodiment, the first and second epitopes are on different antigens, e.g., the different proteins (or different subunits of a multimeric protein). In another embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In a further embodiment, a bispecific antibody molecule comprises an antibody having binding specificity for a first epitope and an antibody having binding specificity for a second epitope. In one embodiment, a bispecific antibody molecule comprises an antibody, or fragment thereof, having binding specificity for a first epitope and a half antibody, or fragment thereof, having binding specificity for a second epitope. In one embodiment, a bispecific antibody or fragment thereof comprises a Fab having binding specificity for a first epitope and a Fab having binding specificity for a second epitope.

Also within the scope of the invention are scFv formats.

In another embodiment, the heterodimeric protein, for example the Fc polypeptide or antibody or fragment thereof comprises a further moiety.

For example, the heterodimeric protein is an Fc receptor fusion protein.

The Fc region engages with a family of receptors referred to as the Fcγ receptors (FcγRs). All FcγRs interact with a very similar binding site on Fc. It is known that the constant regions, and in particular the C-regions within the Fc fragment, are responsible for various effector functions of immunoglobulin. Variable regions on the other hand mediate antigen specificity. Effector functions are thus mediated by the Fc fragment and not by the variable regions that provide antigen specificity. Binding to FcγRs elicits a number of responses, for example antibody-dependent cell-mediated cytotoxicity or phagocytosis. Modified Fc regions for veterinary use are described, for example in WO 2019/035010 incorporated herein by reference. Fc modifications as for example described in WO 2019/035010 can thus be combined with the heterodimerisation modifications described herein and proteins that have such combined modifications are within the scope of this invention. Such modifications include amino acid modification relative to a wild-type IgG Fc which result in e.g. including increased Protein A binding (e.g., for ease of purification), decreased CIq binding (e.g., for reduced complement-mediated immune responses), decreased CD16 binding (e.g., for reduced antibody-dependent cellular cytotoxicity (ADCC) induction, increased stability, and/or the ability to form heterodimeric proteins.

There are three classes of human Fcγ receptors (FcγR) which allow IgG to interact with cells of the immune system. In addition, the neonatal FcRn plays a role in placental transport of IgG and in preventing IgG degradation therefore prolonging the half life of circulating IgG. The various types of FcγR have different cellular distributions and their properties, including details of functionally significant polymorphisms. FcvγIR (high affinity), FcγRIIIa (intermediate affinity) and FcγRIIa and FcγRIIIb (low affinity) are all activating receptors and engagement of these receptors can lead to inflammatory reactions including the destruction of target cells. In contrast, the low affinity FcγRIIb is the only inhibitory FcvIR and, as such, is of special interest. FcγRIIb is found on B cells, where it acts to prevent activation and proliferation, on mast cells and basophils and on macrophages, monocytes and neutrophils, where it can inhibit target cell destruction.

For example, the Fc polypeptide as described herein may be combined with a binding domain capable of specifically binding to a target molecule. Thus, in aspect, the invention relates to fusion proteins comprising a Fc region as described herein. In the fusion protein, one or more polypeptide is operably linked to an Fc region of the invention. For example, the Fc domain may be linked to a Fab binding domain. The binding domain may comprise more than one polypeptide chain in association e.g. covalent or otherwise (e.g. hydrophobic interaction, ionic interaction, or linked via sulphide bridges).

Modifications described herein can also be combined with any other Fc modification e.g. with modified effector function.

Generally, the one or more polypeptide operably linked to an Fc region of the invention may be any protein or small molecule, for example a binding domain derived from any molecule with specificity for another molecule and capable of binding said molecule. The binding domain will have an ability to interact with a target molecule which will preferably be another polypeptide, but may be any target (e.g. carbohydrate, lipid (such as phospholipid) or nucleic acid). Preferably, the interaction will be specific. Typically, the target will be antigen present on a cell, or a receptor with a soluble ligand. This may be selected as being a therapeutic target, whereby it is desired to bind it with a molecule having the properties discussed above. The target may be present on or in a target cell, for example a target cell which it is desired to lyse, or in which it is desired to induce apoptosis. Protein fusion partners may thus include, but are not limited to, the variable region of any antibody, the target-binding region of a receptor, an adhesion molecule, a ligand, an enzyme, a cytokine, an antigen, a chemokine, or any other protein or protein domain. Small molecule fusion partners may include any therapeutic agent that directs the Fc fusion to a therapeutic target. Such targets may be any molecule, preferably an extracellular receptor, which is implicated in disease.

The invention also relates to the use of the Fc as defined herein in a method for making a Fc fusion protein. For example, this can be used to create heterodimers with extended half life by having Fc fusion.

In another embodiment, the antibody of the invention comprises a further moiety. For example, the moiety is a half life extending moiety, e.g. a canine or caninized serum albumin or a variant thereof. The antibody may also be modified to increase half-life, for example by a chemical modification, especially by PEGylation, or by incorporation in a liposome.

Half-life may be increased by at least 1.5 times, preferably at least 2 times, such as at least 5 times, for example at least 10 times or more than 20 times, greater than the half-life of the corresponding antibody without the half life extension. For example, increased half-life may be more than 1 hours, preferably more than 2 hours, more preferably more than 6 hours, such as more than 12 hours, or even more than 24, 48 or 72 hours, compared to the corresponding antibody without the half life extending moiety.

In another embodiment, the moiety is a therapeutic moiety, such as a drug, an enzyme or a toxin. In one embodiment, the therapeutic moiety is a toxin, for example a cytotoxic radionuclide, chemical toxin or protein toxin. Thus, the invention also encompasses an immunoconjugate, e.g. an antibody conjugated (joined) to a second molecule, usually a toxin, radioisotope or label. These conjugates are used in immunotherapy and to develop monoclonal antibody therapy as a targeted form of chemotherapy when they are often known as antibody-drug conjugates (ADCs).

The toxic payload may be selected from small molecules (e.g. maytansanoid, auristatin), a protein toxin (e.g. Pseudomonas exotoxin, diphtheria toxin), a cytolytic immunomodulatory protein (e.g. Fas ligand) to kill targeted cells, a biologically active peptide (e.g. GLP-1) to extend the pharmacological half-life of the natural peptide, an enzymes (e.g. urease) to modify the biochemistry of the targeted microenvironment or radionuclides (e.g. 90Y, 111 In) for either killing or imaging of tumor cells.

In another embodiment, the antibody is labelled with a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

The moiety can be linked to the heterodimeric protein, e.g. antibody, using linkers known in the art, e.g. via a chemical or peptide linker. The linkage can be covalent or non-covalent. An exemplary covalent linkage is via a peptide bond. In some embodiments, the linker is a polypeptide linker (L). Suitable linkers include for example a linker with GS residues such as (Gly4Ser)n wherein n=1-50, e.g. 1 to 10. Other linkage/conjugation techniques include cysteine conjugation, e.g. for cysteine-based site-specific antibody conjugation to a toxic payload.

In another aspect, the invention relates to a polypeptide comprising a canine IgG CH3 domain wherein said polypeptide comprises an amino acid substitution at position K414 in IgG-D or at a corresponding position in IgG-A, B or C with a negatively charged amino acid or an amino acid substitution at position E424 in IgG-D or at a corresponding position in IgG-A, B or C with a positively charged amino acid. In one embodiment, the polypeptide comprises an Fc domain. In another aspect, the invention relates to a polypeptide comprising a canine IgG CH3 domain wherein said polypeptide comprises an amino acid substitution at one or more of position 424, 423, 378, 414, 416, 433, 392, 383, 393, 383 or 378 in IgG-D or at a corresponding position in IgG-A, B or C with an amino acid of the opposite charge and/or an amino acid substitution at 436 in IgG-D or at a corresponding position in IgG-A, B or C with a charged amino acid or with an amino acid of the opposite charge and/or an amino acid substitution at 425 in IgG-D or at a corresponding position in IgG-A, B or C with N.

For example, with reference to IgG-B, the polypeptide may comprise a canine IgG CH3 domain wherein said polypeptide comprises an amino acid substitution at one or more of position 428, 427, 382, 383, 420, 418, 437, 467, 386, 397, 396, 429 with an amino acid of the opposite charge or a substitution at 429 or 387.

Thus, with reference to IgG-B, In one embodiment of the polypeptide, the substitution at E428 is E428K, E428R or E428H, the substitution at D427 is D427K, D427R or D427H, the substitution at E382 is E382K, E382R or E382H, the substitution at E383 is E383K, E383R, E383H or E383R, the substitution at R420 is R420E or R420D, the substitution at K418 is K418E or K418D, the substitution at K437 is K437D or K437E, the substitution at K467 is K467D or K467E, the substitution at K396 is K396E or K396D, the substitution at K386 is K386E or K386D, the substitution at D397 is D397K, D397R or D397H, the substitution at K387 is K387D or K387E and the substitution at D429 is D429N.

In one embodiment, the substitution at E428 is E428K, the substitution at D427 is D427K, the substitution at E382 is E382K, the substitution at E383 is E383K, the substitution at R420 is R420D, the substitution at K418 is K418E, the substitution at K437 is K437D, the substitution at K467 is K467E, the substitution at K396 is K396E, the substitution at K386 is K386D, the substitution at D397 is D397K, the substitution at K387 is K387D and the substitution at D429 is D429N.

Specific substitutions that can be made in the polypeptide are set out in the mutation IDs in Tables 1 and 2 and are also described above with respect to the heterodimeric polypeptide.

In one embodiment, the polypeptide is a canine, caninized or canine derived heavy chain. In one embodiment, the polypeptide comprises one or more further mutation in the CH3 domain. Exemplary mutations are detailed above. The polypeptide is preferably an isolated polypeptide.

In another aspect, the invention relates to a nucleic acid molecule encoding the heterodimeric protein or encoding the polypeptide of the invention. The nucleic acid is preferably an isolated nucleic acid.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature.

Furthermore, we provide an isolated nucleic acid construct comprising at least one nucleic acid as defined above. The construct may be in the form of a plasmid, vector, transcription or expression cassette. Thus, the invention also relates to a plasmid, vector, transcription or expression cassette comprising a nucleic acid of the invention. Expression vectors of use in the invention may be constructed from a starting vector such as a commercially available vector. After the vector has been constructed and a nucleic acid molecule encoding a heavy chain, or a light chain and a heavy chain sequence has been inserted into the proper site of the vector, the completed vector may be inserted into a suitable host cell for amplification and/or polypeptide expression.

The invention also relates to an isolated recombinant host cell comprising one or more nucleic acid molecule plasmid, vector, transcription or expression cassette as described above. The transformation of an expression vector into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

The host cell may be eukaryotic or prokaryotic, for example a bacterial, viral, plant, fungal, mammalian or other suitable host cell. In one embodiment, the cell is an E. coli cell. In another embodiment, the cell is a yeast cell. In another embodiment, the cell is a Chinese Hamster Ovary (CHO) cell, HeLa cell or other cell that would be apparent to the skilled person. Mammalian cell lines available as hosts for expression are well known in the art and include, but are not limited to, immortalized cell lines available from the American Type Culture Collection (ATCC) and any cell lines used in an expression system known in the art can be used to make the recombinant polypeptides of the invention.

In general, host cells are transformed with a recombinant expression vector that comprises DNA encoding a desired bispecific antibody. Among the host cells that may be employed are prokaryotes, yeast or higher eukaryotic cells. Prokaryotes include gram negative or gram-positive organisms, for example E. coli or bacilli. Higher eukaryotic cells include insect cells and established cell lines of mammalian origin. Examples of suitable mammalian host cell lines include the COS-7 cells, L cells, 0127 cells, 3T3 cells, Chinese hamster ovary (CHO) cells, or their derivatives and related cell lines which grow in serum free media, HeLa cells, BHK cell lines, the CVIIEBNA cell line, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays.

Other suitable host cells include insect cells, using expression systems such as baculovirus in insect cells, plant cells, transgenic plants and transgenic animals, and by viral and nucleic acid vectors.

Alternatively, it is possible to produce the polypeptide in lower eukaryotes such as fungal cell lines and yeast or in prokaryotes such as bacteria. Suitable yeasts include S. cerevisiae, S. pombe, Kluyveromyces strains, Pichia pastoris, Candida, or any yeast strain capable of expressing heterologous polypeptides. Suitable bacterial strains include E. coli, B. subtilis, S. typhimurium, or any bacterial strain capable of expressing heterologous polypeptides. If the bispecific antibody is made in yeast or bacteria, it may be desirable to modify the product produced therein, for example by phosphorylation or glycosylation of the appropriate sites, in order to obtain a functional product. Such covalent attachments can be accomplished using known chemical or enzymatic methods.

A host cell, when cultured under appropriate conditions, can be used to express bispecific antibody that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

A skilled person will know that there are different ways to identify, obtain and optimise the heterodimeric proteins as described herein, including in vitro and in vivo expression libraries. Optimisation techniques known in the art, such as display (e.g., ribosome and/or phage display) and/or mutagenesis (e.g., error-prone mutagenesis) can be used.

In such a method, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition, Brian K. Kay, Jill Winter, John McCafferty, 1996).

Libraries, for example phage libraries, are generated by isolating a cell or tissue expressing an antibody or heavy chain, cloning the sequence encoding the genes from mRNA derived from the isolated cell or tissue and displaying the encoded protein using a library. The heavy chain can be expressed in mammalian, bacterial, yeast or other expression systems.

Therefore, in another aspect, the invention also relates to an expression library comprising a plurality of polypeptides or antibodies as described herein. Phage display library screening is advantageous over some other screening methods due to the vast number of different polypeptides (typically exceeding 10⁹) that can be contained in a single phage display library. This allows for the screening of a highly diverse library in a single screening step. In general, the libraries include repertoires of V genes (e.g., harvested from populations of lymphocytes or assembled in vitro) which are cloned for display of associated heavy and light chain variable domains on the surface of filamentous bacteriophage. Phages are selected by binding to an antigen. Soluble antibodies are expressed from phage infected bacteria and the antibody can be improved, such as, by mutagenesis. Methods of producing antibodies by making, screening and evolving antibodies and antibody libraries are established.

In a further aspect, the invention relates to a method for making a heterodimeric protein or a polypeptide comprising the steps of

a) transforming a host cell with a nucleic acid of or a vector as described herein;

b) culturing the host cell and expressing first and second IgG CH3 containing polypeptides and

c) recovering the heterodimeric protein or polypeptide from the host cell culture.

A heterodimeric protein or a polypeptide obtained or obtainable by the method is also within the scope of the invention.

Bispecific antibodies of the invention based on the IgG format, comprising of two heavy and two light chains can be produced by a variety of methods known in the art. For instance, bispecific antibodies may be produced by fusing two antibody-secreting cell lines to create a new cell line or by expressing two antibodies in a single cell using recombinant DNA technology. These approaches yield multiple antibody species as the respective heavy chains from each antibody may form monospecific dimers (also called homodimers), which contain two identical paired heavy chains with the same specificity, and bispecific dimers (also called heterodimers) which contain two different paired heavy chains with different specificity. In addition, light chains and heavy chains from each antibody may randomly pair to form inappropriate, non-functional combinations. This problem, known as heavy and light chain miss-pairings, can be solved by choosing antibodies that share a common light chain for expression as bispecifics. Methods to address the light chain-heavy chain mispairing problem include the generation of bispecific antibodies using a single light chain. This requires heavy-light chain engineering or novel antibody libraries that utilize a single light chain that limits the diversity. In addition, antibodies with a common light chain have been identified from transgenic mice with a single light chain. Another approach is to swap the CH1 domain of one heavy chain with CL domain of its cognate light chain (Crossmab technology). Also covered are scFv formats.

In another aspect, there is provided a pharmaceutical composition comprising a heterodimeric molecule of the invention and optionally a pharmaceutically acceptable carrier. A heterodimeric protein or pharmaceutical composition described herein can be administered by any convenient route, including but not limited to oral, topical, parenteral, sublingual, rectal, vaginal, ocular, intranasal, pulmonary, intradermal, intravitrial, intratumoural, intramuscular, intraperitoneal, intravenous, subcutaneous, intracerebral, transdermal, transmucosal, by inhalation, or topical, particularly to the ears, nose, eyes, or skin or by inhalation. In another embodiment, delivery is of the nucleic acid encoding the drug, e.g. a nucleic acid encoding the molecule of the invention is delivered.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the heterodimeric protein of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition can be in the form of a liquid, e.g., a solution, syrup, solution, emulsion or suspension. The liquid can be useful for oral administration or for delivery by injection, infusion (e.g., IV infusion) or sub-cutaneously.

When intended for oral administration, the composition can be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by intravenous injection or infusion.

The amount of the heterodimeric protein or pharmaceutical composition described herein that is effective/active in the treatment of a particular disease or condition will depend on the nature of the disease or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disease, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of a heterodimeric protein of the present invention by weight of the composition. When intended for oral administration, this amount can be varied to range from about 0.1% to about 80% by weight of the composition. Preferred oral compositions can comprise from about 4% to about 50% of the heterodimeric protein of the present invention by weight of the composition.

Compositions can be prepared so that a parenteral dosage unit contains from about 0.01% to about 2% by weight of the heterodimeric protein of the present invention.

For administration by injection, the composition can comprise from about typically about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks.

The invention also relates to therapeutic treatment applications of the heterodimeric protein, e.g. antibody or fragment thereof, as described herein.

As used herein, “treat”, “treating” or “treatment” means inhibiting or relieving a disease or disease. For example, treatment can include a postponement of development of the symptoms associated with a disease or disease, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., canine patients, being treated. Many medical treatments are effective for some, but not all, patients that undergo the treatment.

The term “subject” or “patient” refers to an animal which is the object of treatment, observation, or experiment, suitably a companion animal, specifically a canine.

The invention also relates to a heterodimeric protein or pharmaceutical composition described herein for use in the treatment or prevention of a disease.

In another aspect, the invention relates to the use of a heterodimeric protein or pharmaceutical composition described herein in the treatment or prevention of a disease. In another aspect, the disclosure relates to the use of a heterodimeric protein or pharmaceutical composition described herein in the manufacture of a medicament for the treatment or prevention of a disease as listed herein.

In one embodiment, the heterodimeric protein, e.g. an antibody or fragment thereof, binds to a therapeutic target. This can be a tumor associated antigen (TAA). Tumor antigens can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), posttranslationally altered (tumor-associated alterations in glycosylation, etc.), or idiotypic (highly polymorphic genes where a tumor cell expresses a specific “clonotype”, i.e., as in B cell, T cell lymphoma/leukemia resulting from clonal aberrancies).

In one embodiment, the tumor associated antigen is selected from PSMA, Her2, Her3, CD123, CD19, CD20, CD22, CD23, CD74, BCMA, CD30, CD33, CD52, EGRF, CECAM6, CAXII, CD24, ETA, MAGE, Mesothelin, cMet, TAG72, MUC1, MUC16, STEAP, EphvIII, FAP, GD2, IL-13Ra2, L1-CAM, PSCA, GPC3, gpA33, CA-125, gangliosides G(D2), G(M2) and G(D3), Ep-CAM, CEA, bombesin-like peptides, PSA, HER2/neu, epidermal growth factor receptor (EGFR), erbB2, erbB3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R, TAG-72 and CO17-1A.

In yet another embodiment, the heterodimeric protein, e.g. an antibody or fragment thereof, inhibits tumor cell growth and/or proliferation through binding to its antigen.

In one embodiment, the heterodimeric protein, e.g. an antibody or fragment thereof an inhibitor of an immune checkpoint molecule. This may be selected from an inhibitor of one or more of PD-1, PD-L1, PD-L2, CTLA-4, TIM-3, CEACAM, VISTA, BTLA, TIGIT, LAIR1, CD160, 2B4 or TGFR beta. In another embodiment, the antibody may be an activator of a costimulatory molecule selected, for example, from an agonist of one or more of OX40, OX40L, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, CD3, CD8, CD28, CD4 or ICAM-1.

In yet another embodiment, the heterodimeric protein, e.g. an antibody or fragment thereof binds to a cytokine, e.g. interleukins, such as interleukin-17, interleukin-4, interleukin-13 or interleukin-31, a key cytokine involved in the itching and inflammation associated with atopic dermatitis.

In another embodiment, the target is a tumor necrosis factor (TNF), e.g. TNF alpha.

In another embodiment, the target is a nerve growth factor (NGF) and/or the NGF receptor, therapeutic targets in the treatment of acute and chronic pain states NGF.

In one embodiment, the target is GnRH which is used in immunocastration.

Bispecific heterodimeric proteins, e.g. an antibody or fragment thereof, as described herein that bind to two different targets selected form the targets above, for example an immune checkpoint molecule and another target, a TAA and another target or a cytokine and another target. In one embodiment, the heterodimeric protein, e.g. an antibody or fragment thereof, binds to two different immune checkpoint molecules, two different TAAs or a TAA and immune checkpoint molecule.

The disease treatable with the heterodimeric protein, e.g. an antibody or fragment thereof as described herein, or the pharmaceutical composition, can be selected from a cancer, an immune disease, neurological disease, inflammatory disease, allergy, transplant rejection, viral infection, immune deficiency, an autoimmune disease and other immune system-related disease.

In one embodiment, the protein may be used to treat pain, for example by targeting NGF.

In one embodiment, the treatment is immunocastration, for example by targeting anti-GnRH.

The cancer can be selected from a solid or non-solid tumor. For example, the cancer may be selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, breast cancer, brain cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, lung cancer, non-small cell lung cancer, thymoma, prostate cancer, mesothelioma, adrenocortical carcinoma, lymphomas, such as such as B cell lymphoma, Hodgkin's disease, non-Hodgkin's, gastric cancer, leukemias such as ALL, CLL, AML, urothelial carcinoma leukemia and multiple myelomas.

In one embodiment, the tumor is a solid tumor. Examples of solid tumors which may be accordingly treated include breast carcinoma, lung carcinoma, colorectal carcinoma, pancreatic carcinoma, glioma and lymphoma, such as T-cell lymphomas caused by feline leukemia virus (FeLV). Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include CNS, neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma. Examples of vascularized skin cancers for which the antagonists of this invention are effective include squamous cell carcinoma, basal cell carcinoma and skin cancers that can be treated by suppressing the growth of malignant keratinocytes, such as veterinary malignant keratinocytes.

In one embodiment, the tumor is a non-solid tumor. Examples of non-solid tumors include leukemia, multiple myeloma and lymphoma.

In one embodiment, the cancer is locally advanced unresectable, metastatic, or recurrent cancer.

Cancers include those whose growth may be inhibited using the antibodies of the invention include cancers typically responsive to immunotherapy. Non-limiting examples of preferred cancers for treatment include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer).

In one embodiment, for the treatment of cancer, the protein may target CD3. Optionally, the protein may be provided in the format of a bispecific T-cell engagers (BiTE).

In one embodiment, the cancer has progressed after another treatment, for example chemotherapy.

In another embodiment, the disease is selected from an autoimmune disease, inflammatory conditions, allergies and allergic conditions, hypersensitivity reactions, severe infections, and organ or tissue transplant rejection. The disease may be selected from the following non-limiting list: psoriasis, systemic lupus erythematosis, rheumatoid arthritis, osteoarthritis, juvenile chronic arthritis, spondyloarthropathies, systemic sclerosis, idiopathic inflammatory myopathies, Sjogren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain Barre syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, gluten-sensitive enteropathy, and Whipple's disease, autoimmune or immune-mediated skin diseases including bullous skin diseases, erythema multiforme and contact dermatitis, allergic diseases such as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, autoimmune haematological disorders (including e.g., hemolytic anaemia, aplastic anaemia, pure red cell anaemia and idiopathic thrombocytopenia), autoimmune inflammatory bowel disease (including e.g., ulcerative colitis, Crohn's disease and Irritable Bowel Syndrome), transplantation associated diseases including graft rejection and graft-versus-host-disease, or any veterinary equivalents thereof.

In one embodiment, the heterodimeric protein or pharmaceutical composition described herein is used in combination with an existing therapy or therapeutic agent, for example an anti-cancer therapy. Thus, in another aspect, the invention also relates to a combination therapy comprising administration of a heterodimeric protein or pharmaceutical composition of the invention and an anti-cancer therapy. The anti-cancer therapy may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include other checkpoint inhibitors, antineoplastic agents, immunogenic agents, attenuated cancerous cells, tumor antigens, antigen presenting cells such as dendritic cells pulsed with tumor-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumor-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy. In one embodiment, the heterodimeric protein is used in combination with surgery.

In one embodiment, the heterodimeric protein or pharmaceutical composition as described herein is administered together with an immunomodulator, a checkpoint modulator, an agent involved in T-cell activation, a tumor microenvironment modifier (TME) or a tumour-specific target. For example, the immunomodulator can be an inhibitor of an immune checkpoint molecule selected from an inhibitor of one or more of PD-1, PD-L1, PD-L2, CTLA-4, TIM-3, CEACAM, VISTA, BTLA, TIGIT, LAIR1, CD160, 2B4 or TGFR beta. In another embodiment, the immunomodulator can be an activator of a costimulatory molecule selected from an agonist of one or more of OX40, OX40L, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand, CD3, CD8, CD28, CD4 or ICAM-1.

In a specific embodiment of the present invention, the heterodimeric protein or composition is administered concurrently with a chemotherapeutic agent or with radiation therapy. In another specific embodiment, the chemotherapeutic agent or radiation therapy is administered prior or subsequent to administration of the composition of the present invention, preferably at least an hour, five hours, 12 hours, a day, a week, a month, more preferably several months (e.g. up to three months), prior or subsequent to administration of composition of the present invention.

In some embodiments, the heterodimeric protein or pharmaceutical composition described herein may be administered with two or more therapeutic agents. In some embodiments, the heterodimeric protein or pharmaceutical composition may be administered with two or more therapeutic agents.

The heterodimeric protein or pharmaceutical composition described herein may be administered at the same time or at a different time as the other therapy or therapeutic compound or therapy, e.g., simultaneously, separately or sequentially.

In another aspect, the invention provides a kit for the treatment or prevention of a disease, diagnosis, prognosis or monitoring disease comprising a heterodimeric protein or pharmaceutical composition of the invention. Such a kit may contain other components, packaging and/or instructions.

The kit may include a labeled heterodimeric protein or pharmaceutical composition as described herein and one or more compounds for detecting the label.

The invention in another aspect provides a heterodimeric protein or pharmaceutical composition as described herein of the invention packaged in lyophilized form or packaged in an aqueous medium.

In another aspect, a heterodimeric protein as described herein is used for non-therapeutic purposes, such as diagnostic tests and assays.

Further aspects and embodiments of the invention will be apparent to those skilled in the art given the present disclosure including the following experimental exemplification.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including any references to gene accession numbers and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further described in the non-limiting examples and non-limiting clauses.

### EXAMPLES

### Example 1

### Example 1 CH3/CH3 Interface Charge Pair Identification of Dog IgG Isoforms by Modelling

Several methods have been applied to engineer human IgGs to enrich heavy chain CH3-CH3 heterodimerisation over contaminant monospecific dimers. These IgG heavy chain heterodimerisation methods opened up the generation of bi- and multi-specific antibodies. There are two main methods to promote heterodimerisation using isotype and species matched IgG heavy chain, namely “knob-in-hole” (kiH) by Genentech (WO9627011) and electrostatic steering by Chugai and Amgen (WO2006/106905).

Methods for canine bi/multi specific antibody pairing have not yet been tested.

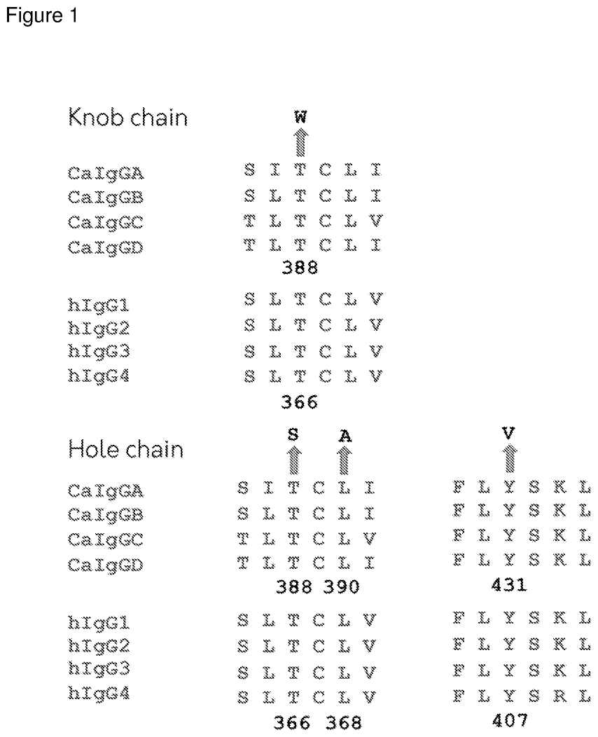

To explore the possibility of such mechanisms for canine IgG, structural based homology modelling is conducted using human IgG dimeric structure (PDB database: 1I6x) as a guide to explore the canine CH3-CH3 interface. We have found that the canine equivalent positions of human KiH modifications are conserved between all four canine and human isotypes. These residues lie within the central hydrophobic core of the CH3-CH3 interface [FIG. 1].

To explore electrostatic steering based mechanism, charged residues at the interface of canine IgG dimer are mapped based on experimentally defined human CH3-CH3 interface (Gunasekaran et al, supra).

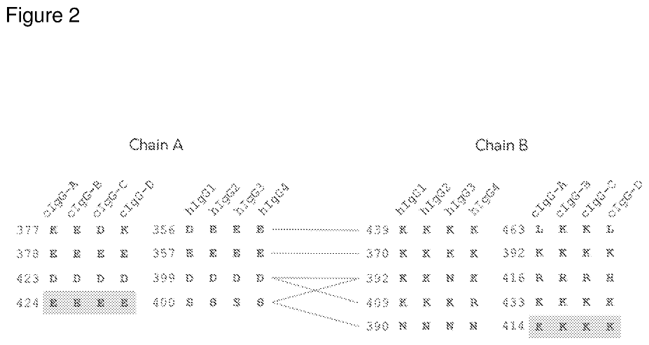

In addition to discovering that most residues forming charge pairs are conserved between human and canine, this analysis highlights the presence of a canine specific charge pair [FIG. 2 shaded in grey]. This charge pair is highly conserved across all canine IgG isotypes and are uncharged in the corresponding positions in all human IgG isotypes [FIG. 2].

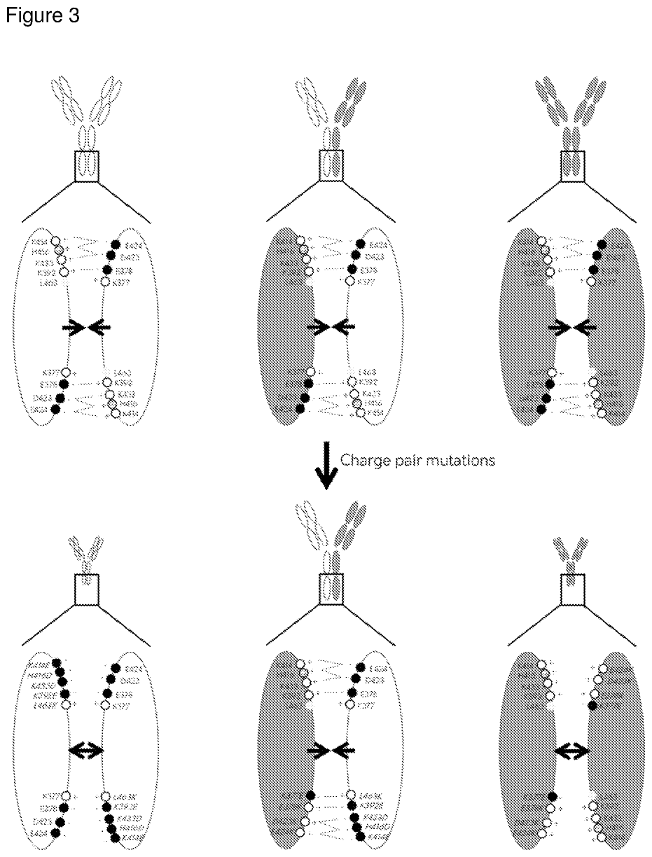

Amino acid mutations to invert charges at these positions should create charge based heterodimeric attraction and homodimer repulsion, therefore enriching for the proportion of heterodimer assembly [FIG. 3]. A series of Canine IgG Chain A and Chain B amino acid substitutions including both electrostatic steering and KiH are summarised in Tables 1 and 2, and the methods for generating these point mutations are described in Example 2.

### Example 2 Construction of Bispecific Chimeric Antibody Expression Vector

Dog genomic DNA is extracted from blood of the Beagle breed (Envigo). Dog genomic DNA is used as template to PCR amplify the constant regions of dog IgG-A, IgG-B, IgG-C and IgG-D using Q5 high fidelity DNA polymerase (New England Biolabs). PCR primers are designed based on publicly available sequence information from Basenji dog (IgG-A, IgG-B, IgG-C, IgG-D; GenBank #SDHF01000364), and, where available, Beagle dog WGS sequences (GenBank accession nos. AOCS01185922, AOCS01019635, AOCS01019304, AOCS01185926) and are used to amplify the corresponding regions from Beagle DNA. The PCR fragments are cloned into TOPO cloning vector pCR-Blunt using Zero Blunt PCR cloning Kit (Invitrogen). Individual clones are sequence verified by Sanger sequencing. Alternatively, canine IgG Fc is synthesised by GeneArt (Thermo Fisher) with the amino acid sequence codon optimised for CHO cell expression.

To be able to distinguish heterodimers from homodimer contaminants by size, two chimeric heavy chains are generated with distinct molecular weight. One heavy chain is generated by fusing OKT3-ScFv to hinge-CH2-CH3 regions of dog IgG-B and IgG-D isoforms, while the other chain is generated to be Fc only. Specifically, OKT3-ScFV and dog IgG-B and IgG-D hinge-CH2-CH3 regions are PCR amplified using Q5 high fidelity DNA polymerase and assembled using NEBuilder HIFI DNA Assembly (New England Biolabs) to generate a ScFv-Fc. The ScFv-Fc (or “scFv-OKT3+Fc-B”) and Fc only (or “Fc-B”) heavy chains were subsequently cloned into mammalian expression vectors PetML5 (Hygromycin resistant) and petML6 (Blasticidin resistant) respectively. Both PetML5 and PetML6 expression vectors consist of CAG promoter and bgh-pA to drive the expression of the cloned insert, which in this case are the modified ScFv-Fc or Fc. For selection in cells, pgk-hph-pgk-pA cassette is present in PetML5 vector to confer Hygromycin resistance, while pgk-bsr-pgk-pA cassette is present in PetML6 vector to confer Blasticidin resistance. Both the expression and drug resistant cassettes are flanked by piggyBac inverted terminal repeats (ITRs), so that upon co-transfection with piggyBac transposase, the sequence flanked by piggyBac ITRs can be stably integrated into the mammalian host cells, in this case, CHO cells. The plasmid vector backbone contains ampicillin resistance cassette for bacterial selection and ColE1 bacterial origin of replication. The heavy chain and the antibiotic resistant gene expression units are flanked by DNA transposon piggyBac terminal inverted repeats to mediate stable integration into host cells in the presence of piggyBac transposase.

A series of mutations, as shown in Tables 1 and 2 are subsequently generated by site directed mutagenesis using a method described by Liu H & Naismith J (BMC Biotechnology 2008, 8:91). All mutations are verified by Sanger Sequencing. KiH mutations are introduced into human IgG4PE (FIG. 1) as a control.

### Example 3 Production of Chimeric Antibody

CHO-S cells are cultured in suspension in Erlenmeyer shaker flasks in FreeStyle F17 CHO Expression media supplemented with L-glutamine and anti-clumping agent (Thermo Fisher Scientific). The suspension cells are cultured in humidified incubator at 37° C. with 8% CO2 shaking at 150 rpm. The two plasmid DNA containing heterodimer heavy chain pairs together with the piggyBac transposase containing plasmid are co-transfected into CHO-S cells using PEIMax with a DNA to PEIMax ratio of 1 ug DNA to 3 ul PEIMax at 1 mg/ml concentration per 1×10⁶ CHO cells. Different ratios of ScFv-Fc and Fc constructs are tested as indicated in the Figures. Stable integration is selected using hygromycin and Blasticidin 24 hours post transfection. After 6-8 days drug selection, the cells were recovered in non-selective media prior to production. For production, 2×10⁶ CHO cells are seeded per 1 ml of media in 32° C. with 8% CO2 shaking at 150 rpm. Cells are fed with L-Glucose, Cell Boost 7a and Cell Boost 7b every 3 days. Cell viability is monitored to be at least over 70% during the production period and at day 12, supernatant is harvest for protein A purification.

### Example 4 Heterodimerisation Evaluation

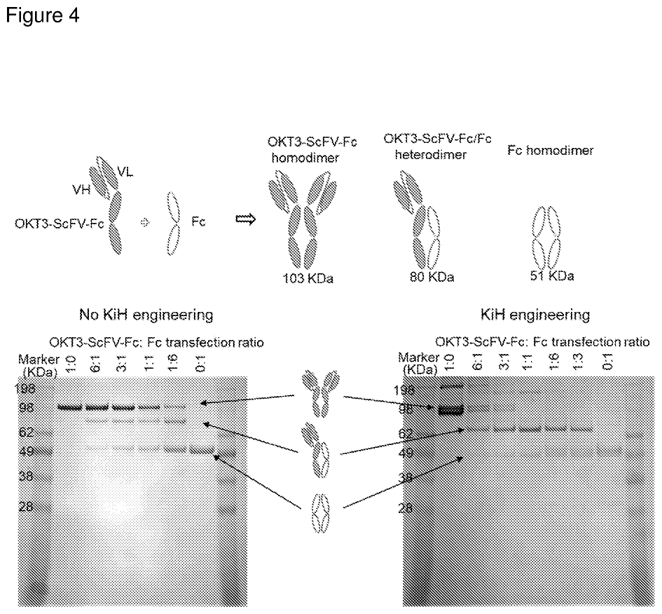

The evaluate the optimal ratio of two chimeric heavy chains for transfection, plasmids encoding human IgG4PE Fc or ScFv-Fc with engineered KiH mutations (Fc-knob and ScFv-hole) are transfected in CHO-S cells at ratios of 1:0, 6:1, 3:1, 1:1, 1:3, 1:6, 0:1. Human IgG4PE without KiH mutations ScFv-Fc and Fc chains with the same transfection ratios are used as controls. Protein A purified ScFv-Fc and Fc assembly are evaluated by non-reducing SDS gel electrophoresis. The ratio of 1:1 produced the most heterodimer and least monomer contaminants (FIG. 4).

To test all elected dog IgG-D ScFv-FC and Fc heavy chain pairs (Table 1), DNA plasmids containing both chains are transfected at 1:1 ratio and stable integration is selected as described. Protein A purified ScFv-Fc and Fc assembly are examined using non-reducing SDS gel electrophoresis. The proportion of heterodimer and homodimer are analysed by ion exchange chromatography. Thermostability and aggregation are also examined.

To test all elected dog IgG-B Fc and ScFv-FC chain pairs (Table 2), DNA plasmids containing both chains are transfected at 1:1, 1:3 or 1:6 ratios and stable integration is selected as described. Protein A purified homo/hetero ScFv-Fc and Fc assembly are quantified using UV spectroscopy (Nanodrop 1000, Thermo Fisher Scientific), normalised to 0.25 mg/mL and 2.5 ug total protein have been loaded on both non-reducing SDS gel electrophoresis and HPLC Size Exclusion Chromatography (HSEC). Heterodimers will migrate or elute differently in both SDS-PAGE and HSEC and are quantified and compared to WT molecules. “Fc-B” MW is 28 KDa so forms a homodimer ˜56 KDa; “scFv-OKT3+Fc-B” is 55 KDa so forms a homodimer ˜110 KDa; and “Fc-B:scFv-OKT3-Fc-B” heterodimer is˜83 KDa. A schematic of homo and heterodimer formation is shown in FIG. 4.

SDS-PAGE was stained using Instant Blue (InstantBlue® Coomassie Protein Stain (ISB1L) (ab119211)) standard protocol. ImageJ software (https://imagej.nih.gov/ij/) is used to quantify SDS-PAGE bands by densitometry. Briefly, a rectangle selection for each lane is generated and the bands' peaks are integrated using implemented ImageJ plugin. Area under each peak (homo/heterodimers) have been plotted.

HPLC-SEC chromatography (column: BioResolve SEC mAb 200A, 2.5 um column, Waters Corporation) was performed using ACQUITY H-class Bio (Waters Corporation) using PBS as mobile phase with isocratic 0.575 mL/min flow rate. Test samples are centrifuged 5′ at 20000 g (using standard table top centrifuge) to remove any precipitates. Empower software is used to integrate each chromatography peaks detected. Percentage of heterodimeric species and Area (indicative of antibody concentration) was determined for each molecule and compared to a WT molecules.

Results from SDS-PAGE analyses of 1:1 scFv-Fc:Fc DNA ratio (FIGS. 7, 8 and 9; mutations as shown in Table 2 were used) show that many mutants improved heterodimer formation in comparison to WT IgG B sequence. In a few combinations expression of one or even both chains is low or absent, suggesting that the introduced mutations could have affected expression, stability and/or folding of the polypeptide chains. It is also worth noting that in addition to increase % of heterodimers, many combinations also showed a single band with MW corresponding to heterodimer, in contrast to at least 3 bands seen in the WT IgG B sequence. This may be indicative of a more stable conformation induced by these mutations. Moreover, the presence of monomeric Fc (that migrates around 28 KDa i.e. observed in mutant ID 23 and 26) is also indicative of homodimer repulsion, which can be helpful during further purification steps in large scale manufacture of a clinical product.

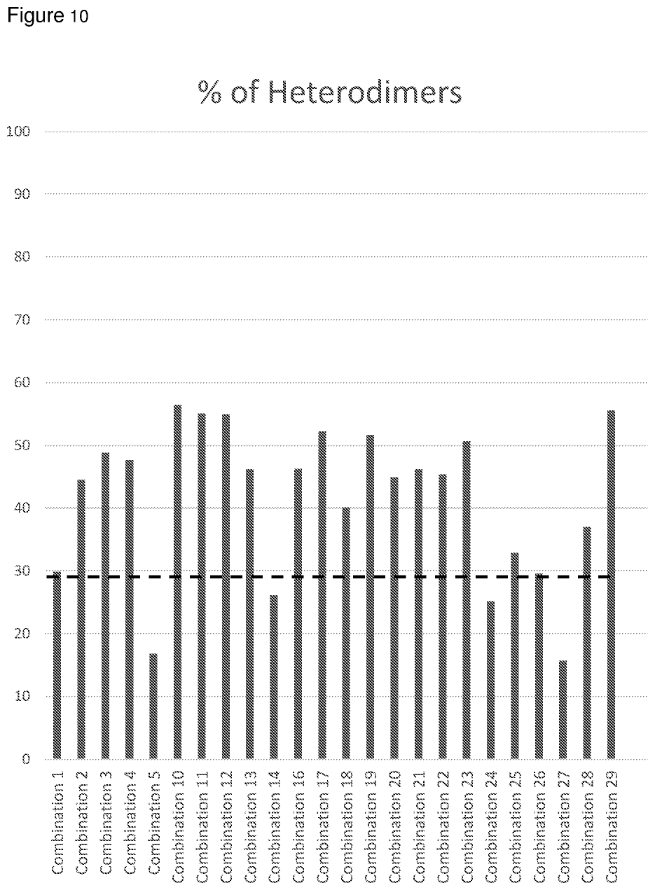

Densitometric analysis to present the % of heterodimers of those expressed proteins is shown in FIG. 10 (mutations as shown in Table 2).

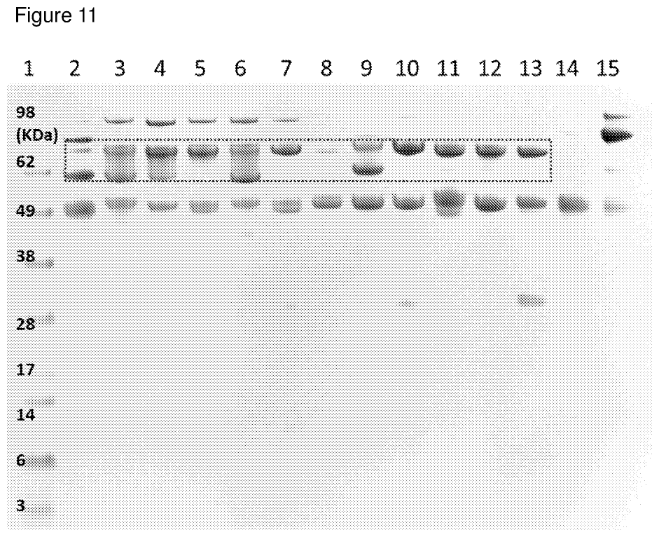

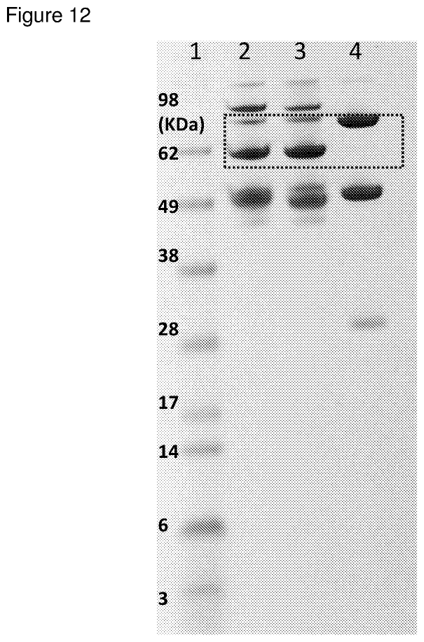

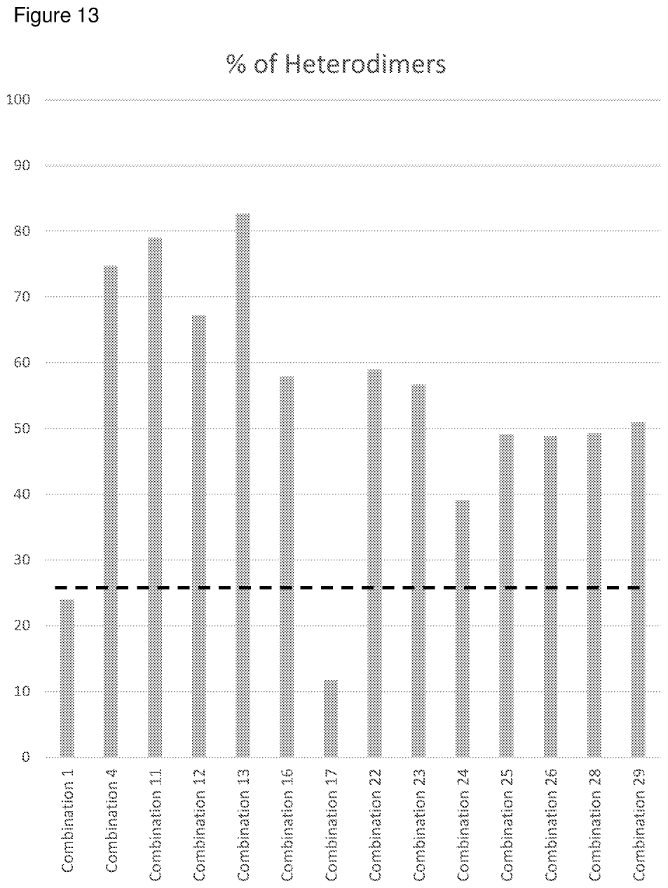

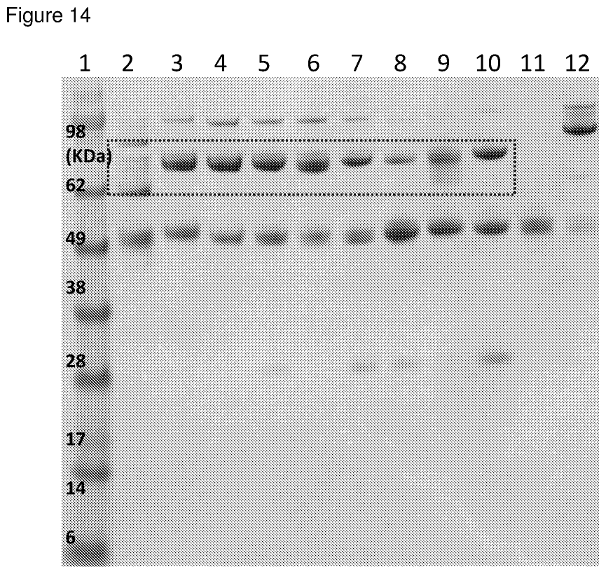

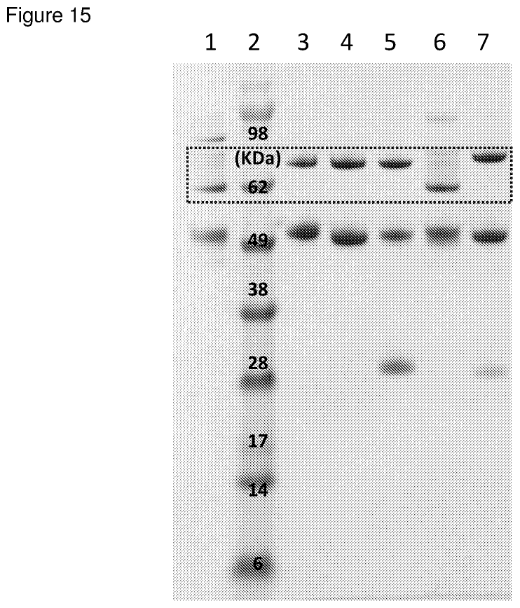

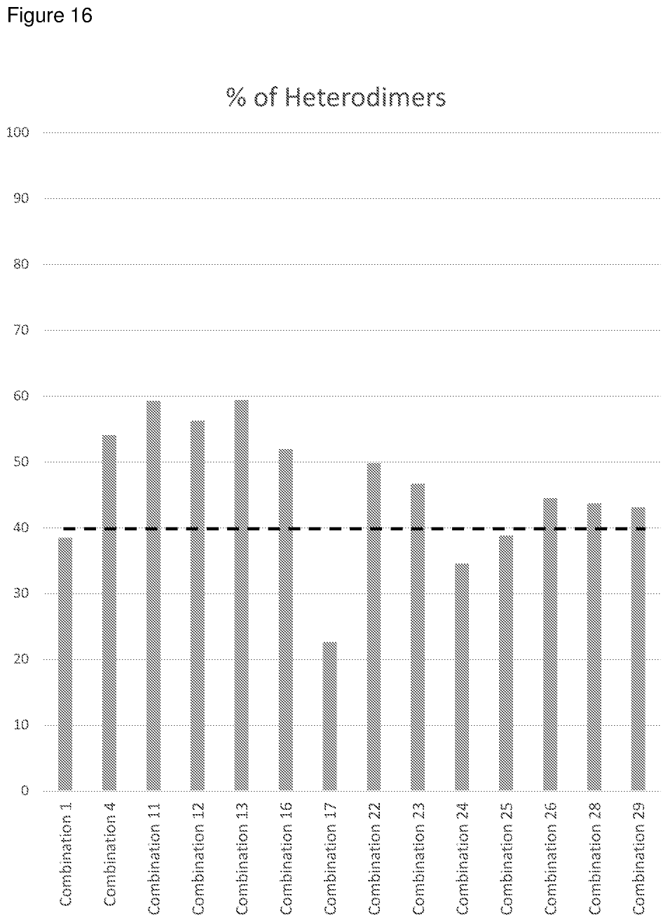

Based on 1:1 scFv-Fc:Fc DNA ratio SDS-PAGE results, another two sets of transfections using 1:3 and 1:6 scFv-Fc:Fc DNA ratio in CHO-s cell line have been performed on selected candidates (ID 4, 11, 12, 13, 16, 17, 22, 23, 24, 25, 26, 28 and 29). Results of the 1:3 transfection ratio are shown in FIGS. 11, 12 and 13; results of the 1:6 transfection ratio are shown in FIGS. 14, 15 and 16. Mutations as shown in Table 2 were used.

In these conditions, mutants also showed an increase in heterodimers in comparison to WT (IgG B) control and showed a similar phenotype for presence of single heterodimer band as well as monomeric Fc.

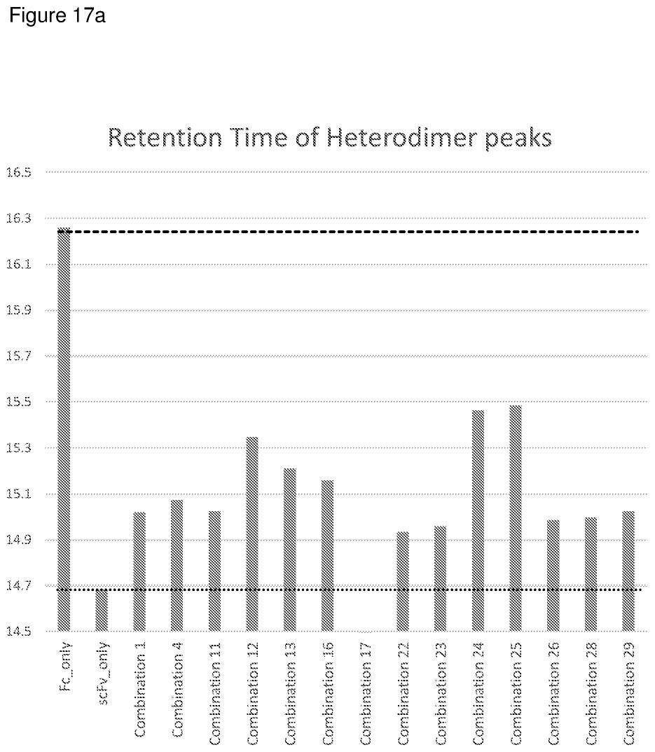

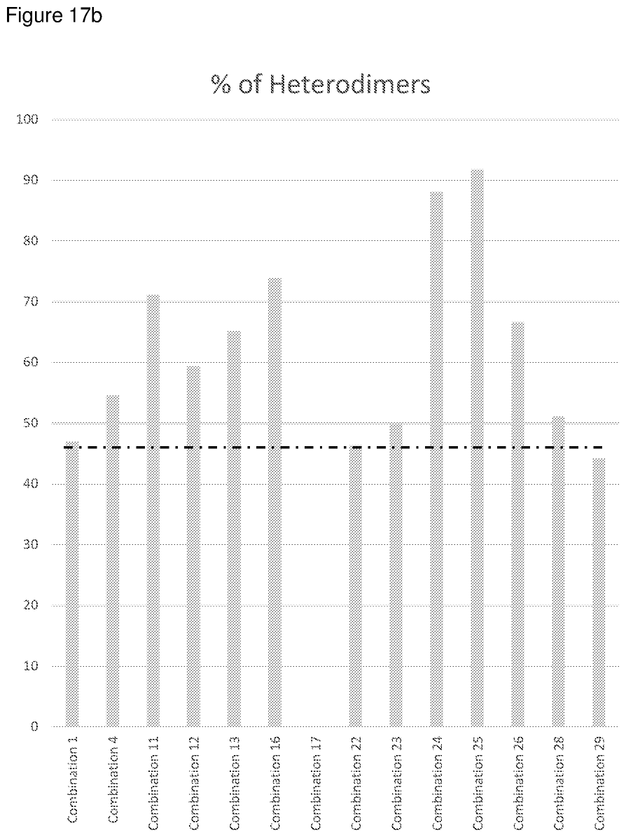

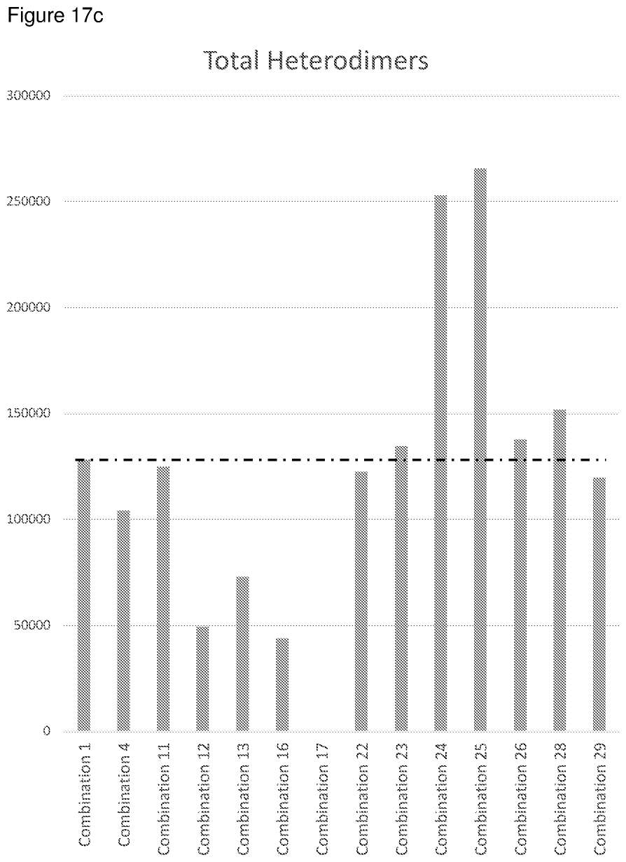

FIG. 17a shows the retention time of heterodimer peaks as measured by HSEC analysis. FIG. 17b shows -HPLC SEC quantification of heterodimers peak shown as % of total protein. This showed similar results as SDS-PAGE quantification, with almost all mutants increasing % of heterodimers. FIG. 17c shows HPLC SEC quantification of heterodimers peak shown as total area. All mutants showed an increase in the percentage of heterodimers peak, apart from ID 17, 22 and 29. All mutations show similar stability as the WT control apart from Mutations IDs (Combinations) 12, 13, 16 and 17 that showed less stable heterodimers and 24, 25 that showed increased stability.

A summary table from the data obtained from the experiments using a 1:3 scFv-Fc:Fc DNA ratio experiment has been generated (FIG. 18).

Further analytical methods to assess % of heterodimers are carried out using ion exchange chromatography (H-SCX (strong cation exchange)). This method allows separation of molecules based on their charges, particularly useful when testing the effect of these mutation in a standard IgG format, where molecular weight is less useful to discriminate between heterodimer identification/separation. This is useful for guiding purification steps during manufacture of bispecific antibodies, for example.

Heterodimer stability in an IgG format can also be assessed by performing accelerated stability test using both temperature and pH as stress factors. HSEC and HSCX are then be used to identify and quantify aggregation/fragmentation propensity as well as the presence of protein variants in order to select most stable combinations.

TABLE 1
Summary of charge pair mutations for heterodimer formation.
Charge pair mutations in canine IgGD

| Mutation/ | | |
|---|---|---|
| combination ID | Chain 1 | Chain 2 |
| 1 | E424K | K414E |
| 2 | E424K | K414E, H416D |
| 3 | D423K | K433D |
| 4 | D423K | K433D, H416D |
| 5 | D423K, E424K | K414E, K433D |
| 6 | D423K, E424K | K414E, K433D, H416D |
| 7 | — | L463K |
| 8 | E378K | K392E |
| 9 | E424K | K414E, H416D, L463K |
| 10 | D423K | K433D, H416D, L463K |
| 11 | E424K | K414E, L463E |
| 12 | D423K | K433D, L463E |
| 13 | D423K, E424K | K414E, K433D, L463E |
| 14 | E424K, K377E | K414E, L463K |
| 15 | D423K, K377E | K433D, L463K |
| 16 | D423K, E424K, K377E | K414E, K433D, L463K |
| 17 | E378K | K392E |
| 18 | D393K | K377D |
| 19 | E378K, D393K | K392E, K377D |
| 20 | K377E | L463K |
| 21 | S382K | — |
| 22 | D383N | — |
| 23 | S382K, D393N | — |
| 24 | S382D | D393K |
| 25 | — | D425N |
| 26 | S382D | D393K, D425N |
| 27-KiH | T388S, L390A, Y431V | T388W |

TABLE 2
Summary of charge pair mutations for heterodimer formation
(numbering related to IgG B).
Charge pair mutations in canine IgGB

| Mutation/ | | |
|---|---|---|
| Combination ID | Chain 1 | Chain 2 |
| 1 | WT | WT |
| 2 | E428K | R420D |
| 3 | E428K | R420D, K418E |
| 4 | D427K | R420D |
| 5 | D427K | R420D, K437D |
| 6 | D427K, E428K | K437D, K418D |
| 7 | D427K, E428K | R420D |
| 8 | D427K, E428K | R420D, K437D |
| 9 | D427K, E428K | K420D, K418D |
| 10 | E382K | K467E |
| 11 | E383K | K396E |
| 12 | E382K, D427K | K418E, R420D |
| 13 | E382K, E428K | K418E, R420D |
| 14 | E382K, D427K | K437D, R420D |
| 15 | E382K, E428K | K437D, R420D |
| 16 | E383K, D427K | K418E, R420D |
| 17 | E383K, E428K | K418E, R420D |
| 18 | E383K, D427K | K437D, R420D |
| 19 | E383K, E428K | K437D, R420D |
| 20 | E428K | K418E, K467E |
| 21 | D427K | K437D, K467E |
| 22 | E428K | K418E, K396E |
| 23 | D427K | K437D, K396E |
| 24 | D427K, E428K | R420D, K467E |
| 25 | D427K, E428K | K396E, K467E |
| 26 | D427K, E428K | K437D, K418E, K467E |
| 27 | D427K, E428K | K437D, K418E, K396E |
| 28 | E428K | K418E |
| 29 | D427K | K437D |
| 30 | K386D | D397K |
| 31 | — | D429N |
| 32 | K386D | D397K, D429N |
| 33 | N387D | — |

1. A heterodimeric protein comprising a first polypeptide comprising a first canine IgG CH3 domain and a second polypeptide comprising a second canine IgG CH3 domain wherein said IgG is selected from IgG-A, B, C or D and wherein
a) said first canine IgG CH3 domain comprises an amino acid substitution at one or more of position 428, 427, 382, 383, 386 in IgG-B or at a corresponding position in IgG-A, C or D with an amino acid of the opposite charge and said second canine IgG CH3 domain comprises an amino acid substitution at one or more of position 420, 418, 437, 467, 396, 397, 429 in IgG-B or at a corresponding position in IgG-A, C or D with an amino acid of the opposite charge or
b) said second canine IgG CH3 domain comprises an amino acid substitution at 429 in IgG-B or at a corresponding position in IgG-A, C or D and the first canine IgG CH3 domain does not comprise a corresponding mutation or
c) said first canine IgG CH3 domain comprises an amino acid substitution at 387 in IgG-B or at a corresponding position in IgG-A, C or D and the second IgG CH3 domain does not comprise a corresponding mutation.

2. The heterodimeric protein according to claim 1 wherein
a) said first canine IgG CH3 domain comprises an amino acid substitution at one or more of position E428, D427, E382, E383, K386 with an amino acid of the opposite charge and said second canine IgG CH3 domain comprises an amino acid substitution at one or more of position R420, K418, K437, K467, K396, D397, D429 with an amino acid of the opposite charge or
b) said second canine IgG CH3 domain comprises an amino acid substitution at D429 and the first canine IgG CH3 domain does not comprise a corresponding mutation or
c) said first canine IgG CH3 domain comprises an amino acid substitution at N387 and the second IgG CH3 domain does not comprise a corresponding mutation.

3. The heterodimeric protein according to claim 1 or 2 wherein said first canine IgG CH3 domain comprises an amino acid substitution at position E428 with a positively charged amino acid and said second canine IgG CH3 domain comprises an amino acid substitution at position R420 with a negatively charged amino acid.

4. The heterodimeric protein according to claim 1 or 2 wherein said first canine IgG CH3 domain comprises an amino acid substitution at position E428 with a positively charged amino acid and said second canine IgG CH3 domain comprises an amino acid substitution at position R420 with a negatively charged amino acid and an amino acid substitution at position K418 with a negatively charged amino acid.

5. The heterodimeric protein according to claim 1 or 2 wherein said first canine IgG CH3 domain comprises an amino acid substitution at position D427 with a positively charged amino acid and said second canine IgG CH3 domain comprises an amino acid substitution at position R420 with a negatively charged amino acid.

6. The heterodimeric protein according to claim 1 or 2 wherein said first canine IgG CH3 domain comprises an amino acid substitution at position D427 with a positively charged amino acid and said second canine IgG CH3 domain comprises an amino acid substitution at position R420 with a negatively charged amino acid and an amino acid substitution at position K437 with a negatively charged amino acid.

7. The heterodimeric protein according to claim 1 or 2 wherein said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position K437 with a negatively charged amino acid and an amino acid substitution at position K418 with a negatively charged amino acid.

8. The heterodimeric protein according to claim 1 or 2 wherein said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position R420 with a negatively charged amino acid.

9. The heterodimeric protein according to claim 1 or 2 wherein said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position R420 with a negatively charged amino acid and an amino acid substitution at position K437 with a negatively charged amino acid.

10. The heterodimeric protein according to claim 1 or 2 wherein said first canine IgG CH3 domain comprises an amino acid substitution at position D427 with a positively charged amino acid and an amino acid substitution at position E428K with a positively charged amino acid and said second canine IgG CH3 domain comprises an amino acid substitution at position K420 with a negatively charged amino acid and an amino acid substitution at position K418 with a negatively charged amino acid.

11. The heterodimeric protein according to claim 1 or 2 wherein said first canine IgG CH3 domain comprises an amino acid substitution at position E382 with a positively charged amino acid amino acid and said second canine IgG CH3 domain comprises an amino acid substitution at position K467 with a negatively charged amino acid.

12. The heterodimeric protein according to claim 1 or 2 wherein said first canine IgG CH3 domain comprises an amino acid substitution at position E383 with a positively charged amino acid amino acid and an amino acid substitution at position K396 with a negatively charged amino acid.

13. The heterodimeric protein according to claim 1 or 2 wherein said first canine IgG CH3 domain comprises an amino acid substitution at position E382 with a positively charged amino acid and an amino acid substitution at position D427 with a positively charged amino acid and said second canine IgG CH3 domain comprises an amino acid substitution at position K418 with a negatively charged amino acid and at position R420 with a negatively charged amino acid.

14. The heterodimeric protein according to claim 1 or 2 wherein said first canine IgG CH3 domain comprises an amino acid substitution at position E382 with a positively charged amino acid and an amino acid substitution at position E428 with a positively charged amino acid and said second canine IgG CH3 domain comprises an amino acid substitution at position K418 with a negatively charged amino acid and at position R420 with a negatively charged amino acid.

15. The heterodimeric protein according to claim 1 or 2 wherein said first canine IgG CH3 domain comprises an amino acid substitution at position E382 with a positively charged amino acid and an amino acid substitution at position D427 with a positively charged amino acid and said second canine IgG CH3 domain comprises an amino acid substitution at position K437 with a negatively charged amino acid and at position R420 with a negatively charged amino acid.

16. The heterodimeric protein according to claim 1 or 2 wherein said first canine IgG CH3 domain comprises an amino acid substitution at position E382 with a positively charged amino acid and an amino acid substitution at position E428 with a positively charged amino acid and said second canine IgG CH3 domain comprises an amino acid substitution at position K437 with a negatively charged amino acid and at position R420 with a negatively charged amino acid.

17. The heterodimeric protein according to claim 1 or 2 wherein said first canine IgG CH3 domain comprises an amino acid substitution at position E383 with a positively charged amino acid and an amino acid substitution at position D427 with a positively charged amino acid and said second canine IgG CH3 domain comprises an amino acid substitution at position K418 with a negatively charged amino acid and at position R420 with a negatively charged amino acid.

18. The heterodimeric protein according to claim 1 or 2 wherein said first canine IgG CH3 domain comprises an amino acid substitution at position E383 with a positively charged amino acid and an amino acid substitution at position E428 with a positively charged amino acid and said second canine IgG CH3 domain comprises an amino acid substitution at position K418 with a negatively charged amino acid and at position R420 with a negatively charged amino acid

19. The heterodimeric protein according to claim 1 or 2 wherein said first canine IgG CH3 domain comprises an amino acid substitution at position E383 with a positively charged amino acid and an amino acid substitution at position D427 with a positively charged amino acid and said second canine IgG CH3 domain comprises an amino acid substitution at position K437 with a negatively charged amino acid and at position R420 with a negatively charged amino acid.

20. The heterodimeric protein according to claim 1 or 2 wherein said first canine IgG CH3 domain comprises an amino acid substitution at position E383 with a positively charged amino acid and an amino acid substitution at position E428 with a positively charged amino acid and said second canine IgG CH3 domain comprises an amino acid substitution at position K437 with a negatively charged amino acid and at position R420 with a negatively charged amino acid.

21. The heterodimeric protein according to claim 1 or 2 wherein said first canine IgG CH3 domain comprises an amino acid substitution at position E428 with a positively charged amino acid and said second canine IgG CH3 domain comprises an amino acid substitution at position K418 with a negatively charged amino acid and at position K467 with a negatively charged amino acid

22. The heterodimeric protein according to claim 1 or 2 wherein said first canine IgG CH3 domain comprises an amino acid substitution at position D427 with a positively charged amino acid and said second canine IgG CH3 domain comprises an amino acid substitution at position K437 with a negatively charged amino acid and at position R467 with a negatively charged amino acid

23. The heterodimeric protein according to claim 1 or 2 wherein said first canine IgG CH3 domain comprises an amino acid substitution at position E428 with a positively charged amino acid and said second canine IgG CH3 domain comprises an amino acid substitution at position K418 with a negatively charged amino acid and at position K396 with a negatively charged amino acid.

24. The heterodimeric protein according to claim 1 or 2 wherein said first canine IgG CH3 domain comprises an amino acid substitution at position D427 with a positively charged amino acid and said second canine IgG CH3 domain comprises an amino acid substitution at position K437 with a negatively charged amino acid and at position K396 with a negatively charged amino acid.

25. The heterodimeric protein according to claim 1 or 2 wherein said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position R420 with a negatively charged amino acid and at position K467 with a negatively charged amino acid.

26. The heterodimeric protein according to claim 1 or 2 wherein said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position K396 with a negatively charged amino acid and at position K467 with a negatively charged amino acid.

27. The heterodimeric protein according to claim 1 or 2 wherein said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position K437 with a negatively charged amino acid, at position K418 with a negatively charged amino acid and at position K467 with a negatively charged amino acid.

28. The heterodimeric protein according to claim 1 or 2 wherein said first canine IgG CH3 domain comprises an amino acid substitution at position D427 with a positively charged amino acid and an amino acid substitution at position E428 with a positively charged amino acid and said second canine IgG CH3 domain comprises an amino acid substitution at position K437 with a negatively charged amino acid, at position K418 with a negatively charged amino acid and at position K396 with a negatively charged amino acid.

29. The heterodimeric protein according to claim 1 or 2 wherein said first canine IgG CH3 domain comprises an amino acid substitution at position E428 with a positively charged amino acid and said second canine IgG CH3 domain comprises an amino acid substitution at position K418 with a negatively charged amino acid.

30. The heterodimeric protein according to claim 1 or 2 wherein said first canine IgG CH3 domain comprises an amino acid substitution at position D427 with a positively charged amino acid and said second canine IgG CH3 domain comprises an amino acid substitution at position K437 with a negatively charged amino acid.

31. The heterodimeric protein according to claim 1 or 2 wherein said first canine IgG CH3 domain comprises an amino acid substitution at position K386 with a negatively charged amino acid and said second canine IgG CH3 domain comprises an amino acid substitution at position D397 with a positively charged amino acid.

32. The heterodimeric protein according to claim 1 or 2 wherein said first canine IgG CH3 domain does not comprises an amino acid substitution and said second canine IgG CH3 domain comprises an amino acid substitution at position D429.

33. The heterodimeric protein according to claim 1 or 2 wherein said first canine IgG CH3 domain comprises an amino acid substitution at position K386 with a negatively charged amino acid and said second canine IgG CH3 domain comprises an amino acid substitution at position D397 with a positively charged amino acid and a substitution at D429.

34. The heterodimeric protein according to claim 1 or 2 wherein said first canine IgG CH3 domain comprises an amino acid substitution at position N387 with a negatively charged amino acid and said second canine IgG CH3 domain does not comprise an amino acid substitution.

35. The heterodimeric protein according to a preceding claim wherein the substitution at E428 is E428K, E428R or E428H, the substitution at D427 is D427K, D427R or D427H, the substitution at E382 is E382K, E382R or E382H, the substitution at E383 is E383K, E383R, E383H or E383R, the substitution at R420 is R420E or R420D, the substitution at K418 is K418E or K418D, the substitution at K437 is K437D or K437E, the substitution at K467 is K467D or K467E, the substitution at K396 is K396E or K396D, the substitution at K386 is K386E or K386D, the substitution at D397 is D397K, D397R or D397H, the substitution at D429 is D429N, the substitution at N387 is N387D.

36. The heterodimeric protein according to a preceding claim wherein said heterodimeric protein comprises an amino acid modification in chain 1 and chain 2 as set out in Table 2.

37. The heterodimeric protein according to a preceding claim wherein said protein comprises one or more further amino acid substitution in the first canine IgG CH3 domain and/or in the second canine IgG CH3 domain.

38. The heterodimeric protein according to any preceding claim wherein said heterodimeric protein comprises a Fc region.

39. The heterodimeric protein according to any preceding claim wherein said first polypeptide is an antibody heavy chain or fragment thereof and/or said second polypeptide is an antibody heavy chain or fragment thereof.

40. The heterodimeric protein according to any preceding claim wherein said heterodimeric protein comprises a first and a second light chain.

41. The heterodimeric protein according to any preceding claim wherein said heterodimeric protein is an antibody or fragment thereof.

42. The heterodimeric protein according to claim 41 wherein said antibody is a canine or caninized antibody or fragment thereof.

43. The heterodimeric protein according to claim 41 or 42 wherein said fragment is an Fc receptor fusion protein.

44. The heterodimeric protein according to any preceding claim wherein said antibody is a multispecific antibody.

45. The heterodimeric protein according to claim 44 wherein said antibody is a bispecific antibody.

46. The heterodimeric protein according to a preceding claim comprising a further moiety.

47. The heterodimeric protein according to claim 46 wherein said moiety is a half life extending moiety.

48. The heterodimeric protein according to claim 47 wherein said half life extending moiety is canine serum albumin.

49. The heterodimeric protein according to claim 47 wherein said moiety is a cytotoxic moiety.

50. A polypeptide comprising a canine IgG CH3 domain wherein said polypeptide comprises an amino acid substitution at one or more of position 428, 427, 382, 383, 420, 418, 437, 467, 396, 386, 387 or 397 in IgG-B or at a corresponding position in IgG-A, C or D with an amino acid of the opposite charge or an amino acid substitution at D429 with N.

51. A nucleic acid encoding the heterodimeric protein of any of claims 1 to 48 or the polypeptide of claim 50.

52. A vector comprising the nucleic acid of claim 51.

53. A host cell comprising the vector of claim 52.

54. The host cell of claim 53 wherein the host cell is a bacterial, mammalian or yeast cell.

55. A method for making a heterodimeric protein or a polypeptide comprising the steps of d) transforming a host cell with a nucleic acid of claim 51 or a vector of claim 52;
e) culturing the host cell and expressing a first and second IgG polypeptide comprising a CH3 domain and f) recovering the heterodimeric protein or polypeptide from the host cell culture.

56. A pharmaceutical composition comprising a heterodimeric protein according to any of claims 1 to 49 or a polypeptide of claim 50 and a pharmaceutical carrier.

57. A kit comprising a heterodimeric protein according to any of claims 1 to 49 or a polypeptide of claim 50 and optionally instructions for use.


SEQUENCE LISTING (4 sequences)

SEQ ID NO: 1 - protein, 471 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 2 - protein, 475 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 3 - protein, 473 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSQSGSTVALACLVSGYIPEPVTVSWNSGSLTSGVHTFPSILQSSGLYSLSSMVTVPSSRWPSETFTCNVAHPATNTKVDKPVVKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 4 - protein, 471 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASSTAPSVFPLAPSCGSTSGSTVALACLVSGYFPEPVTVSWNSGSLTSGVHTFPSVLKSSGLYSLSSMVTVPSSRLPSETFTCNVVHPATNTKVDKPVPKESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK